(19) United States
(12) Patent Application Publication
(10) Pub. No.: US 2009/0155854 A1
(43) Pub. Date: Jun. 18, 2009

(54) METHOD FOR AMPLIFYING A FLAVIVIRUS cDNA IN A PROKARYOTIC CELL

(75) Inventors: Andrew YUEH, Zhunan Town (TW); Szu-Yuan PU, Zhunan Town (TW); Chi-Chen YANG, Zhunan Town (TW); Yu-Sheng CHAO, Zhunan Town (TW)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: National Health Research Institute (an institution of Taiwan, R.O.C.), Zhunan (TW)

(21) Appl. No.: 11/934,869

(22) Filed: Nov. 5, 2007

Related U.S. Application Data
(60) Provisional application No. 60/864,172, filed on Nov. 3, 2006.

(51) Int. Cl. C12P 19/34; C07H 21/04; C12N 15/63; C12N 1/00; C12N 7/00

(52) U.S. Cl. 435/91.2; 536/23.1; 435/320.1; 435/243; 435/235.1

(57) ABSTRACT

The invention relates to a method for amplifying a functional flavivirus cDNA in a prokaryotic cell, such as E. coli. The method involves a modified flavivirus cDNA that has one or more silent mutations in one or more prokaryotic promoter regions within a flavivirus cDNA. The silent mutation decreases or abolishes the promoter activity from the prokaryotic promoter region without resulting in a change to the amino acid sequence encoded by the modified flavivirus cDNA as compared to that encoded by the flavivirus cDNA. The invention also relates to the functional flavivirus cDNA generated by the method, its complement, and its RNA transcript. The invention further relates to vectors, host cells and flavivirus related to the functional flavivirus cDNA.

### CROSS-REFERENCE TO RELATED APPLICATIONS

This application claims the benefit of Provisional Application No. 60/864,172, filed Nov. 3, 2006, the disclosure of which is incorporated herein by reference.

### BACKGROUND OF THE INVENTION

The present invention generally relates to a genetic manipulation technique, and more particularly, to a method for amplifying a Flavivirus cDNA in a prokaryotic cell.

The Flavivirus genus consists of more than 70 members with different antigenic groups. Most of them are transmitted by mosquitoes or ticks and cause serious human and animal diseases (Monath et al., Fields Virology, 3^{rd} ed., vol. 1, pp. 961-1034). They include, for example, dengue virus (DEN), Japanese encephalitis virus (JEV), West Nile virus (WNV), yellow fever virus (YFV), and tick-borne encephalitis virus (TBE).

A flavivirus is an enveloped RNA virus having a single stranded, positive-sense, 10.5 to 11 kb genomic RNA that is associated with multiple copies of capsid proteins. The genomic RNA is translated into a single polyprotein. As the translated polyprotein enters a host cell, it is then cleaved by both host proteases and a single virus-encoded protease into three structural proteins (C, M and E) and seven non-structural proteins (NS1, NS2A, NS2B, NS3, NS4A, NS4B and NS5) to initiate viral replication in the host cell (Lindenbach et al., Adv. Vir. Res., vol. 59: 23-61 (2003)).

An introduction of flavivirus genomic RNA into susceptible cell lines has resulted in the production of infectious virus particles. This has led to development of a number of methodologies which involve genetically manipulating functional complementary DNA (cDNA) clones to study flavivirus virology. U.S. Pat. No. 6,171,854 and U.S. Pat. No. 6,589,522 to Galler et al. specifically disclosed yellow fever (YF) infectious cDNA and a vaccine composition for humans against YF infection.

Also, recombinant cDNA clones that can be transcribed into full-length infectious RNA provide a powerful tool for studying the virus replication of positive-strand RNA viruses. U.S. Pat. No. 6,794,174 to Pletnev et al. disclosed full-length infectious cDNA clones of Langat tick-borne flavivirus. In the field of flavivirus research, a genetic manipulation of functional complementary DNA (cDNA) clones has provided insights into viral replication and pathogenesis, as well as new strategies in the vaccine development (Ruggli et al., Adv Vir Res, 53: 183-207 (1999)). However, the existing methodologies are unable to resolve the intrinsic toxic properties of flavivirus cDNA sequence in a prokaryotic cell, such as Escherichia coli (E. coli), which result in slow growth of the prokaryotic cell, low yield of flavivirus cDNA and RNA transcripts of the flavivirus with low infectivity.

Until today, little is known about what causes the low production or instability of a flavivirus cDNA in a prokaryotic cell. There remains a need to develop a method to effectively amplify a functional flavivirus cDNA from a prokaryotic cell, such as E. coli.

### BRIEF SUMMARY OF THE INVENTION

It is now discovered that the introduction of one or more silent mutations to one or more prokaryotic promoter regions within a flavivirus cDNA allows amplification of a functional flavivirus cDNA from a prokaryotic cell. The silent mutation decreases or abolishes the promoter activity from the prokaryotic promoter region, thus reducing the cryptic expression of one or more toxic polypeptides from the flavivirus cDNA within the prokaryotic cell, without resulting in a change to the encoded amino acid sequence.

In one general aspect, the present invention relates to a method for amplifying a functional flavivirus cDNA in a prokaryotic cell. The method comprises:

(a) constructing a modified flavivirus cDNA by introducing a silent mutation into a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;

(b) introducing the modified flavivirus cDNA into the prokaryotic cell; and

(c) amplifying the functional flavivirus cDNA by replication of the modified flavivirus cDNA in the prokaryotic cell.

In another aspect, the present invention relates to an isolated nucleic acid molecule selected from the group consisting of:

(i) a modified flavivirus cDNA comprising a silent mutation in a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;

(ii) a complement of the modified flavivirus cDNA; and

(iii) an RNA transcript of the modified flavivirus cDNA.

In other general aspects, the present invention relates to a vector comprising a modified flavivirus cDNA or a complement thereof according to embodiments of the present invention, and a prokaryotic cell comprising the vector.

In a further general aspect, the present invention relates to a flavivirus produced by a host cell transfected with an RNA transcript of the modified flavivirus cDNA according to embodiments of the present invention.

In one embodiment, the present invention relates to a dengue virus type 2 (DEN2), which has a genomic cDNA comprising SEQ ID NO:1 and at least one silent mutation at a nucleotide (nt) region selected from the group consisting of nucleotides 160-205, 198-243, 376-421, 633-678, 1059-1104, 2104-2182, 2582-2627 and 2615-2660 of SEQ ID NO:1. The silent mutation decreases or abolishes the prokaryotic promoter activity of the recited region without resulting in a change to the amino acid sequence encoded by the sequence.

In one embodiment, the present invention relates to a Japanese encephalitis virus (JEV), which has a genomic cDNA comprising SEQ ID NO:2 and at least one silent mutation at a nucleotide (nt) region selected from the group consisting of nucleotides 60-105, 72-117 and 1352-1397 of SEQ ID NO:2. The silent mutation decreases or abolishes the prokaryotic promoter activity of the recited region without resulting in a change to the amino acid sequence encoded by the sequence.

Additional aspects and advantages of the invention will be set forth in part in the description which follows, and in part will be apparent from the description, or can be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a histogram illustrating relative luciferase activity (RLU) expressed by the bacterial strains carrying the DNA fragments from wild-type or mutant DEN2 according to one example of the invention;

FIGS. 2a through to 2c illustrate construction of a full-length functional DEN2 cDNA clone according to an example of the invention; and

FIGS. 3a through to 3d illustrate construction of a full-length functional JEV cDNA clone according to a further example of the invention.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used frequently, which shall have the meanings as set forth in the specification. It must be noted that as used herein and in the appended claims, the singular forms “a,” “an,” and “the” include plural referents unless the context clearly dictates otherwise.

In the context of the present invention, adenosine is abbreviated as “A”, cytidine is abbreviated as “C”, guanosine is abbreviated as “G”, thymidine is abbreviated as “T”, and uridine is abbreviated as “U”.

As used herein, the term “a prokaryotic promoter region” refers to a regulatory region of DNA that is involved in the binding of a prokaryotic RNA polymerase (RNAP) to initiate transcription of a gene inside a prokaryotic cell. Various types of sigma factors, i.e., prokaryotic transcription initiation factors that are part of the RNAP, are involved for specific binding of the RNAP to the promoter to initiate gene transcription. Different sigma factors recognize different promoter sequences. E. coli has at least eight sigma factors; the number of sigma factors varies between bacterial species.

The prokaryotic promoter region often consists of two short sequences at −10 and −35 positions upstream (“5′ to”) from the transcription start site. The sequence at −10 position (−10 element) is essential to start transcription in prokaryotes. The sequence at −35 position (−35 element) allows a high transcription rate. Sigma factor 70, a sigma factor with a molecular weight of 70 kDa, recognizes the consensus sequence SEQ ID NO:82, 5′-TATAAT-3′ at −10 position and the consensus sequence SEQ ID NO: 83, 5′-TTGACA-3′ at −35 position. Both of the consensus sequences, i.e., the most common sequence to appear at such positions, while conserved on average, are not found intact in most promoters. On average only 3 of the 6 base pairs in each consensus sequence are found in any given promoter. Indeed, no promoter has been identified to date that has intact consensus sequences at both the −10 and −35 positions. Some promoters contain so-called “extended-10 element” having a consensus sequence SEQ ID NO: 84, 5′-TGNTATAAT-3′. It should be noted that complexes of prokaryotic RNA polymerase with other sigma factors may recognize different core promoter sequences.

A “reporter gene” refers to a nucleic acid sequence that encodes a reporter gene product. As is known in the art, reporter gene products are typically easily detectable by standard methods. Exemplary suitable reporter genes include, but are not limited to, genes encoding luciferase (lux), β-galactosidase (lacZ), green fluorescent protein (GFP), chloramphenicol acetyltransferase (CAT), β-glucuronidase, neomycin phosphotransferase, and guanine xanthine phosphoribosyl-transferase proteins.

As used herein, “operably linked”, refers to a functional relationship between two nucleotide sequences. A single-stranded or double-stranded nucleic acid moiety comprises the two nucleotide sequences arranged within the nucleic acid moiety in such a manner that at least one of the two nucleotide sequences is able to exert a physiological effect by which it is characterized upon the other. By way of example, a promoter sequence that controls expression (for example, transcription) of a coding sequence is operably linked to that coding sequence. Operably linked nucleic acid sequences can be contiguous, typical of many promoter sequences, or non-contiguous, in the case of, for example, nucleic acid sequences that encode repressor proteins. Within a recombinant expression vector, “operably linked” is intended to mean that the coding sequence of interest is linked to the regulatory sequence(s) in a manner that allows for expression of the coding sequence, e.g., in an in vitro transcription/translation system or in a host cell when the vector is introduced into the host cell.

As used herein, the term “nucleotide sequence,” “nucleic acid” or “polynucleotide” refers to the arrangement of either deoxyribonucleotide or ribonucleotide residues in a polymer in either single- or double-stranded form. Nucleic acid sequences can be composed of natural nucleotides of the following bases: T, A, C, G, and U, and/or synthetic analogs of the natural nucleotides.

As used herein, an “isolated” nucleic acid molecule is one that is substantially separated from at least one of the other nucleic acid molecules present in the natural source of the nucleic acid, or is substantially free of at least one of the chemical precursors or other chemicals when the nucleic acid molecule is chemically synthesized. An “isolated” nucleic acid molecule can also be, for example, a nucleic acid molecule that is substantially free of at least one of the nucleotide sequences that naturally flank the nucleic acid molecule at its 5′ and 3′ ends in the genomic DNA of the organism from which the nucleic acid is derived. A nucleic acid molecule is “substantially separated from” or “substantially free of” other nucleic acid molecule(s) or other chemical(s) in preparations of the nucleic acid molecule when there is less than about 30%, 20%, 10%, or 5% or less, and preferably less than 1%, (by dry weight) of the other nucleic acid molecule(s) or the other chemical(s) (also referred to herein as a “contaminating nucleic acid molecule” or a “contaminating chemical”).

Isolated nucleic acid molecules include, without limitation, separate nucleic acid molecules (e.g., cDNA or genomic DNA fragments produced by PCR or restriction endonuclease treatment, or an RNA transcript produced from an in vitro transcription system or isolated from a cell) independent of other sequences, as well as nucleic acid molecules that are incorporated into a vector, an autonomously replicating plasmid, a virus (e.g., a retrovirus, adenovirus, or herpes virus), or into the genomic DNA of a prokaryote or eukaryote. In addition, an isolated nucleic acid molecule can include a nucleic acid molecule that is part of a hybrid or fusion nucleic acid molecule. An isolated nucleic acid molecule can be a nucleic acid sequence that is: (i) amplified in vitro by, for example, polymerase chain reaction (PCR) or in vitro transcription; (ii) synthesized by, for example, chemical synthesis; (iii) recombinantly produced by cloning; or (iv) purified, as by cleavage and electrophoretic or chromatographic separation.

A polynucleotide can have a single strand or parallel and anti-parallel strands. Thus, a polynucleotide can be a single-stranded or a double-stranded nucleic acid. A polynucleotide is not defined by length and thus includes very large nucleic acids, as well as short ones, such as an oligonucleotide.

A complement of a nucleic acid molecule hybridizes to the nucleic acid molecule under stringent hybridization conditions. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6× sodium chloride/sodium citrate (SSC) at about 45° C., followed by one or more washes in 0.2×SSC and 0.1% SDS at 50-65° C.

Conventional notation is used herein to describe polynucleotide sequences. The left-hand end of a single-stranded polynucleotide sequence is the 5′-end, and the left-hand direction of a single-stranded polynucleotide sequence is referred to as the 5′-direction. The left-hand end of a double-stranded polynucleotide sequence is the 5′-end of the plus strand, which is depicted as the top strand of the double strands, and the right-hand end of the double-stranded polynucleotide sequence is the 5′-end of the minus strand, which is depicted as the bottom strand of the double strands. The direction of 5′ to 3′ addition of nucleotides to nascent RNA transcripts is referred to as the transcription direction. A DNA strand having the same sequence as an mRNA is referred to as the “coding strand.” Sequence on a DNA strand which is located 5′ to a reference point on the DNA is referred to as “upstream sequence,” sequence on a DNA strand which is 3′ to a reference point on the DNA is referred to as “downstream sequence.”

As used herein, “nucleotide X of a nucleotide sequence” refers to the nucleotide that is the Xth residue of the nucleotide sequence counting from its 5′ end. For example, “nucleotide 15 of SEQ ID NO:1” refers to the 15^{th} residue of SEQ ID NO:1 counting from its 5′ end.

As used herein, “recombinant” refers to a polynucleotide, a polypeptide encoded by a polynucleotide, a cell, a viral particle or an organism that has been modified using molecular biology techniques to something other than its natural state.

As used herein, a “recombinant cell” or “recombinant host cell” is a cell that has had introduced into it a recombinant polynucleotide sequence. For example, recombinant cells can contain at least one nucleotide sequence that is not found within the native (non-recombinant) form of the cell or can express native genes that are otherwise abnormally expressed, under-expressed, or not expressed at all. Recombinant cells can also contain genes found in the native form of the cell wherein the genes are modified and re-introduced into the cell by artificial means. The term also encompasses cells that contain an endogenous nucleic acid that has been modified without removing the nucleic acid from the cell; such modifications include those obtained, for example, by gene replacement, and site-specific mutation.

Recombinant DNA sequence can be introduced into host cells using any suitable method including, for example, electroporation, calcium phosphate precipitation, microinjection, transformation, biolistics and viral infection. Recombinant DNA can or can not be integrated (covalently linked) into chromosomal DNA making up the genome of the cell. For example, the recombinant DNA can be maintained on an episomal element, such as a plasmid. Alternatively, with respect to a stably transformed or transfected cell, the recombinant DNA has become integrated into the chromosome so that it is inherited by daughter cells through chromosome replication. This stability is demonstrated by the ability of the stably transformed or transfected cell to establish cell lines or clones comprised of a population of daughter cells containing the exogenous DNA.

Recombinant host cells can be prokaryotic or eukaryotic, including bacteria such as E. coli, fungal cells such as yeast, mammalian cells such as cell lines of human, bovine, porcine, monkey and rodent origin, and insect cells such as Drosophila- and silkworm-derived cell lines. It is further understood that the term “recombinant host cell” refers not only to the particular subject cell, but also to the progeny or potential progeny of such a cell. Because certain modifications can occur in succeeding generations due to either mutation or environmental influences, and in such circumstances, such progeny cannot, in fact, be identical to the parent cell, but are still included within the scope of the term as used herein.

“Sequence” means the linear order in which monomers occur in a polymer, for example, the order of amino acids in a polypeptide or the order of nucleotides in a polynucleotide.

As used herein “silent mutation” refers to a change to the genetic material, e.g., DNA, of an organism that does not result in a change to the amino acid sequence of a polypeptide encoded by the genetic material. A silent mutation can occur in a non-coding region (e.g., outside of a gene or within an intron). A silent mutation can also occur within a coding region (e.g. within an exon) in a manner that does not alter the final amino acid sequence, e.g., by substituting a codon with a degenerative codon for the same amino acid.

“Transformation”, “transform”, and “transformed” denote the process of introducing exogenous DNA into a host cell and the resulting presence in the host cell of the introduced DNA. The term is used broadly to encompass the introduction of a variety of DNA constructs into prokaryotic and eukaryotic cells. Transformation of cultured mammalian cells is commonly referred to as “transfection”.

“Vector” or “construct” refers to a nucleic acid molecule into which a heterologous or isolated nucleic acid can be or is inserted. A vector can be used to deliver the heterologous or isolated nucleic acid to the interior of a cell. Some vectors can be introduced into a host cell allowing for replication of the vector or for expression of a protein that is encoded by the vector or construct. Vectors typically have selectable markers, for example, genes that encode proteins allowing for drug resistance, origins of replication sequences, and multiple cloning sites that allow for insertion of a heterologous sequence. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but not limited to, adenoviral vectors, adeno-associated virus vectors, retroviral vectors, and the like. The properties, construction and use of such vectors, as well as other vectors, in the present invention will be readily apparent to those of skill from the present disclosure.

As used herein, the term “flavivirus cDNA” refers to a complementary DNA (cDNA) that can be synthesized from a flavivirus RNA template in a reaction catalyzed by the enzyme reverse transcriptase. The flavivirus cDNA can be synthesized from a flavivirus RNA template that contains the genetic material encoding a particular protein product of the flavivirus. The flavivirus cDNA can also be synthesized from a flavivirus genomic RNA template that contains the genetic material for the entire flavivirus, i.e., encoding all protein products of the flavivirus. The flavivirus cDNA can be amplified by PCR reaction or by DNA replication in a host cell.

It was observed that when the flavivirus cDNA was amplified in a prokaryotic cell, it imposed certain intrinsic toxicity to the cell, resulting in slow growth of the cell, low yield of flavivirus cDNA and transcripts with low infectivity. It is now discovered that the intrinsic toxicity can be due to the cryptic expression of one or more polypeptides encoded by the flavivirus cDNA in the prokaryotic cell, and that blocking or decreasing the expression of such one or more polypeptides reduced the intrinsic toxicity and resulted in more efficient amplification of functional flavivirus cDNA from a prokaryotic cells.

In one aspect, the present invention is directed to a method for amplifying a functional flavivirus cDNA in a prokaryotic cell. The method comprises:

(a) constructing a modified flavivirus cDNA by introducing a silent mutation into a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;

(b) introducing the modified flavivirus cDNA into the prokaryotic cell; and

(c) amplifying the functional flavivirus cDNA by replication of the modified flavivirus cDNA in the prokaryotic cell.

The genomic flavivirus cDNA may contain several prokaryotic promoter regions. The presence of a prokaryotic promoter region in the flavivirus cDNA can be predicted and verified using methods known in the art in view of the present disclosure. For example, the prokaryotic promoter region can be predicted using various sequence analysis software programs. Because A and T pair together with only two hydrogen bonds (as opposed to three as with G and C), they are easier to break apart, making them favorable sites for RNAPs to latch onto, thus more commonly found in a promoter region. Such predicted prokaryotic promoter region can be isolated from a cell or synthesized in vitro, operably linked to a reporter gene, and assayed for its promoter activity by measuring the reporter gene product in a prokaryotic cell.

To construct the modified flavivirus cDNA, one or more silent mutations are introduced into the one or more prokaryotic promoter regions within the flavivirus cDNA. The silent mutation decreases or abolishes the promoter activity from the prokaryotic promoter region, without resulting in a change to the amino acid sequence encoded by the modified flavivirus cDNA as compared to that encoded by the flavivirus cDNA. Because the silent mutation does not change the amino acid sequence, it does not alter protein function, thus reducing the infectivity of the flavivirus containing the mutation.

Various promoter prediction software can be used to assist the design of silent mutations that can be introduced into a promoter region to decrease or abolish the promoter activity. The prokaryotic promoter regions comprising one or more silent mutations can be operably linked to a reporter gene. The expression level of the reporter gene in a prokaryotic cell indicates whether the prokaryotic promoter activity is abolished or decreased by the silent mutation.

In preferred embodiments, the silent mutation is selected from the group consisting of an A to C substitution, A to G substitution, C to T substitution, T to C substitution and T to G substitution.

The modified flavivirus cDNA can be introduced into a prokaryotic cell by various methods known to the art in view of the present invention. For example, the modified flavivirus cDNA on a vector can be introduced into the prokaryotic cell via methods include, but are not limited to, calcium chloride transformation, electroporation, etc. The vector can be replicated in the prokaryotic cell by DNA replication. In one embodiment of the present invention, the vector is a plasmid. In a preferred embodiment of the present invention, the vector is a multiple copy plasmid, i.e., one that can be replicated and maintained in the prokaryotic cells in multiple copies.

In embodiments of the present invention, the method of the present invention can be used to amplify a function cDNA for any flavivirus in a prokaryotic cell. Such flavivirus includes, but is not limited to, a dengue virus (DEN), Japanese encephalitis virus (JEV), West Nile virus (WNV), yellow fever virus (YFV), and tick-borne encephalitis virus (TBE).

In one embodiment, the method can be used to amplify a functional cDNA for a dengue virus type 2 (DEN-2 or DEN2), such as a DEN2 having a genomic cDNA of SEQ ID NO:1. In a preferred embodiment, the method involves introducing one or more silent mutations to a prokaryotic promoter region within SEQ ID NO:1 selected from the group consisting of nt 160-205, 198-243, 376-421, 633-678, 1059-1104, 2104-2182, 2582-2627 and 2615-2660 of SEQ ID NO:1. In a more preferred embodiment, the method involves introducing one or more silent mutations to SEQ ID NO:1 at a position selected from the group consisting of nt 186, 190, 192, 226, 228, 231, 406, 663, 1093, 1101, 2135, 2612, 2643, 2644 and 2649 of SEQ ID NO:1.

In another embodiment, the method can be used to amplify a functional cDNA for a JEV, such as a JEV having a genomic cDNA of SEQ ID NO:2. In a preferred embodiment, the method involves introducing one or more silent mutations to a prokaryotic promoter region within SEQ ID NO:2 selected from the group consisting of nt 60-105, 72-117 and 1352-1397 of SEQ ID NO:2. In a more preferred embodiment, the method involves introducing one or more silent mutations to SEQ ID NO:2 at a position selected from the group consisting of nt 90, 101, 104, 107 and 1355 of SEQ ID NO:2.

Any prokaryotic cells can be used in methods according to embodiments of the present invention. In a preferred embodiment, the prokaryotic cell is an Escherichia coli cell.

In embodiments of the present invention, two or more silent mutations can be introduced into the modified flavivirus cDNA in order to decrease or abolish the cryptic expression of the toxic polypeptides from the flavivirus cDNA. The two or more silent mutations can be within one prokaryotic promoter region, or within two or more prokaryotic promoter regions, within the flavivirus cDNA.

Another general aspect of the invention relates to an isolated nucleic acid molecule selected from the group consisting of:

(i) a modified flavivirus cDNA comprising a silent mutation in a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;

(ii) a complement of the modified flavivirus cDNA; and

(iii) an RNA transcript of the modified flavivirus cDNA.

In one embodiment of the present invention, the isolated nucleic acid molecule includes the modified flavivirus cDNA, a complement thereof, and an RNA transcript thereof, which are related to a DEN-2 cDNA, such as that which comprises SEQ ID NO:1. In a preferred embodiment, the isolated nucleic acid molecule comprises SEQ ID NO:1 and one or more silent mutations in a prokaryotic promoter region selected from the group consisting of nt 160-205, 198-243, 376-421, 633-678, 1059-1104, 2104-2182, 2582-2627 and 2615-2660 of SEQ ID NO:1, a complement thereof, or a RNA transcript thereof. In a more preferred embodiment, the isolated nucleic acid molecule comprises SEQ ID NO:1 and one or more silent mutations at a position selected from group consisting of nt 186, 190, 192, 226, 228, 231, 406, 663, 1093, 1101, 2135, 2612, 2643, 2644 and 2649 of SEQ ID NO:1, a complement thereof, or a RNA transcript thereof.

In another embodiment of the present invention, the isolated nucleic acid molecule includes the modified flavivirus cDNA, a complement thereof, and an RNA transcript thereof, which are related to the JEV cDNA, such as that which comprises SEQ ID NO:2. In a preferred embodiment, the isolated nucleic acid molecule comprises SEQ ID NO:2 and one or more silent mutations in a prokaryotic promoter region selected from the group consisting of nt 60-105, 72-117 and 1352-1397 of SEQ ID NO:2, a complement thereof, or a RNA transcript thereof. In a more preferred embodiment, the isolated nucleic acid molecule comprises SEQ ID NO:2 and one or more silent mutations at a position selected from group consisting of nt 90, 101, 104, 107 and 1355 of SEQ ID NO:2, a complement thereof, or a RNA transcript thereof.

Methods are known to those skilled in the art to produce an isolated nucleic acid molecule according to embodiments of the present invention in view of the present disclosure. For example, the RNA transcript according to embodiments of the present invention can be produced from an in vitro transcription system. The use of bacteriophage promoters, such as SP6 and T7 polymerase also allows transcription of RNAs with defined 5′ terminal sequences. Working Examples are provided below on how to make and use exemplary isolated nucleic acid molecules according to embodiments of the invention.

RNA transcripts can be assayed for its infectivity by transfection of susceptible host cells, including but not limited to baby hamster kidney fibroblast (BHK21) cells, Aedes albopictus (C6/36) cells, and African green monkey kidney (Vero cell). Transfection can be enhanced by DEAE dextran, cationic liposomes, and electroporation.

The specific infectivity of transcript RNA can be measured and compared to that of RNA extracted from the parental virus, which does not contain the silent mutations, by conducting infectious center assays. Such an assay provides an important index of the quality of a functional clone. Direct assay of infectivity after RNA transfection also provides an early phenotypic comparison with the parental virus with respect to plaque or immunostained focus size, cytopathic effect, or other parameters specific to different members of the flaviviridae. Experiments can be conducted to demonstrate that the virus recovered originates from the cloned cDNA. Experimental evidence for this can be obtained by including various transcription controls (DNase treatment before or after transcription, RNase treatment before or after transcription, etc.) and by engineering genetic markers in the template DNA and showing that these markers are present in the recovered virus. Further analysis of recovered virus can involve examining properties that are important for future genetic studies, such as replication in cell culture, host range, and pathogenesis in animal models. Again, this involves a side-by-side comparison with a parental virus that is used in earlier studies and as the source for cDNA cloning.

Another general aspect of the present invention relates to a vector comprising a modified flavivirus cDNA or a complement thereof according to embodiments of the present invention. Such vectors can be a plasmid that has an origin of replication in a prokaryotic cell. Working Examples are provided below on how to make and use exemplary vectors according to embodiments of the present invention.

The present invention also relates to a prokaryotic cell comprising the vector according to embodiment of the invention. In preferred embodiment, the prokaryotic cell is an E. coli cell.

The present invention further relates to a flavivirus produced by a host cell transfected with an RNA transcript according to embodiments of the invention. The flavivirus can be selected from the group consisting of a dengue virus (DEN), Japanese encephalitis virus (JEV), West Nile virus (WNV), yellow fever virus (YFV), and tick-borne encephalitis virus (TBE).

In one embodiment of the present invention, the flavivirus relates to a DEN-2. In a preferred embodiment, the DEN-2 has a genomic cDNA sequence of SEQ ID NO:1 and one or more silent mutations in a prokaryotic promoter region selected from the group consisting of nt 160-205, 198-243, 376-421, 633-678, 1059-1104, 2104-2182, 2582-2627 and 2615-2660 of SEQ ID NO:1. In a more preferred embodiment, the DEN-2 has a genomic cDNA sequence of SEQ ID NO:1 and one or more silent mutations at a position selected from group consisting of nt 186, 190, 192, 226, 228, 231, 406, 663, 1093, 1101, 2135, 2612, 2643, 2644 and 2649 of SEQ ID NO:1.

In another embodiment of the present invention, the flavivirus relates to JEV. In a preferred embodiment, the JEV has a genomic cDNA sequence of SEQ ID NO:2 and one or more silent mutations in a prokaryotic promoter region selected from the group consisting of nt 60-105, 72-117 and 1352-1397 of SEQ ID NO:2. In a more preferred embodiment, the JEV has a genomic cDNA sequence of SEQ ID NO:2 and one or more silent mutations at a position selected from group consisting of nt 90, 101, 104, 107 and 1355 of SEQ ID NO:2.

According to embodiments of the present invention, silent mutations can be introduced into prokaryotic promoter regions within a flavivirus cDNA to allow more efficient amplification of a functional flavivirus cDNA in a prokaryotic cell, such as E. coli. The amplified flavivirus cDNA can be used to produce an RNA transcript, which can be used to infect a host cell and produce flavivirus with infectivity not significantly reduced as compared to parental flavivirus that does not contain the silent mutations. Thus, the efficient amplification of a functional flavivirus cDNA in a prokaryotic cell allows more efficient production of flavivirus. Methods according to embodiments of the present invention can be used to more efficiently produce flavivirus vaccine candidates for the development of human immunization or vaccine compositions.

It should be noted that genetic manipulations described in the present invention are performed by the commonly used standard protocols accompanied with commercial enzymes according to manufacturer's instructions. Therefore, the present invention is not limited to specific experimental protocols adopted by one skilled in the art.

The invention will now be described in further detail with reference to the following specific, non-limiting examples.

### EXAMPLE 1

### Preparation of Viral RNA and Viral cDNA with Reverse Transcription and PCR

### Cell Lines and Virus Strains

To prepare viral RNAs, DEN2 viruses of Taiwanese PL046 strain or JEV viruses of RP9 strain kindly provided by Dr. C L. Liao (Institute of Biomedical Sciences, National Defense Medical Center, Taiwan) were grown and amplified in the Aedes albopictus C6/36 cells (American Type Culture Collection (ATCC) number CRL-1660). A virus stock was prepared in C6/36 cells by infecting at an appropriate multiplicity of infection (MOI) with RPMI 1640 medium (Invitrogen, Carlsbad, Calif.) containing 2% fetal bovine serum (FBS) (Invitrogen, Carlsbad, Calif.) and incubated at 28° C. until the cytopathic effect occurs. The supernatant was harvested and stored in 20% FBS at −80° C. Virus titers were determined by a plaque-forming assay on the baby hamster kidney fibroblast (BHK21) cells (ATCC number CCL-10).

Plaque Forming Assay

The BHK21 cells were plated and cultured at a density of 2.25×10⁵ cells per well in a 6-well plate, each well containing 1 ml of Dulbecco's modified Eagle's medium (DMEM) (Invitrogen, Carlsbad, Calif.) supplemented with 4.5 g/L glucose and 5% FBS. 0.1 ml of the serially diluted virus solution was added to about 70 to 80% confluent BHK-21 cells. After adsorption for 2 hrs, the virus solution was replaced with either DMEM containing 0.75% methyl cellulose (Sigma, Poole, UK) and 2% FBS for the culture of the DEN-2 infected cells or DMEM containing 1.2% methyl cellulose and 2% FBS for the culture of the JEV infected cells. On the 6^{th} day post infection, the methyl cellulose solution was removed from the wells and the cells were fixed and stained with crystal violet solution (1% crystal violet, 0.64% NaCl and 2% formaldehyde).

### Preparation of Viral RNAs

A viral titer of 200 μl PL046 or RP9 virus (around 10⁶ pfU/ml) was applied in the purification of viral RNA (30 μl) using the Qiagen RNeasy Kit as described in manufacturer's protocol. Viral RNAs were provided as the templates for the reverse transcription (RT) of viral RNAs using the Transcriptor first strand cDNA synthesis kit (Roche Biochemicals, Basel, Switzerland) with a primer PRS313/D2NGC/XbaI-10724R of SEQ ID NO:3 or JEV-1h939R of SEQ ID NO:4 according to manufacturer's protocol. Ten micro liters of the purified viral RNA was preheated to 65° C. for 5 min and then chilled on ice. The reaction mixture contained 10 μl denatured RNAs plus 0.5 mM each dATP, dCTP, dGTP and dTTP; 10 mM dithiothreitol (DTT); 33 U of RNasin (Roche Biochemicals, Basel, Switzerland); 50 U Transcriptor enzyme (Roche Biochemicals, Basel, Switzerland) plus 1× buffer of transcriptor first strand cDNA synthesis kit. The RT products of DEN2 or JEV viral RNAs were provided as templates for the synthesis of viral cDNAs by PCR.

### Preparation of Viral cDNAs

PCRs were set up to amplify cDNA fragments of DEN2 or JEV genome. The cDNA fragments were designed as DenA (nt 1-246 of SEQ ID NO:1), DenB (nt 197-425 of SEQ ID NO:1), DenC (nt 389-684 of SEQ ID NO:1), DenD (nt 648-1107 of SEQ ID NO:1), DenE (nt 1071-2157 of SEQ ID NO:1), DenF (nt 2119-2625 of SEQ ID NO:1), DenG (nt 2589-3249 of SEQ ID NO:1), DenH (nt 2851-4023 of SEQ ID NO:1), DenI (nt 3438-4460 of SEQ ID NO:1), DenJ (nt 4381-5823 of SEQ ID NO:1), DenK (nt 5416-8064 of SEQ ID NO:1), DenL (nt 7760-9024 of SEQ ID NO:1), DenM (nt 8401-10422 of SEQ ID NO:1), DenN (nt 9700-10723 of SEQ ID NO:1) in the DEN2 genome or JEVA1 (nt 1-1352 of SEQ ID NO:2), JEVA2 (nt 1-1967 of SEQ ID NO:2), JEVB (nt 1623-4055 of SEQ ID NO:2), JEVC (nt 3806-6082 of SEQ ID NO:2), JEVD (nt 5861-8048 of SEQ ID NO:2), JEVE (nt 7820-9559 of SEQ ID NO:2), and JEVF (nt 9333-10976 of SEQ ID NO:2) in the JEV genome. Expand Long template PCR kit (Roche Biochemicals, Basel, Switzerland) was used to amplify the variant viral cDNA fragments. The reaction mixture contained 1 μl RT products as a template, 0.4 μM of primers; 0.2 mM each dNTPs; 1× expand log template buffer 1; 3 U of long template enzyme blend in a volume of 50 μl. The reaction mixtures were preheated to 94° C. for 2 min, followed by 27 cycles, with each cycle including 94° C. for 1 min, 60° C. for 1 min, and 68° C. for 1 min before subjected to one final cycle at 72° C. for 10 min.

### EXAMPLE 2

### Prediction of Prokaryotic Promoter Sequences within DEN2 and JEV Genome Sequences

### Construction of Plasmids for Promoter Activity Analysis

The DNA fragments of wild-type DEN2 used in promoter activity analysis were designed as P1 (nt 1-300 of SEQ ID NO:1), P2 (nt 300-600 of SEQ ID NO:1), P3 (nt 600-900 of SEQ ID NO:1), P4 (nt 900-1200 of SEQ ID NO:1), P5 (nt 1200-1500 of SEQ ID NO:1), P6 (nt 1500-1800 of SEQ ID NO:1), P7 (nt 1800-2100 of SEQ ID NO:1), P8 (nt 2100-2400 of SEQ ID NO:1), P9 (nt 2400-2700 of SEQ ID NO:1), and P10 (nt 2700-3100 of SEQ ID NO:1). The DNA fragments from a mutated DEN2, which has eight silent mutations in the genomic cDNA and is amplified efficiently in E. coli, were designated as mP1 (nt 1-300 of SEQ ID NO:1), mP2 (nt 300-600 of SEQ ID NO:1), mP3 (nt 600-900 of SEQ ID NO:1), mP4 (nt 900-1200 of SEQ ID NO:1), mP5 (nt 1200-1500 of SEQ ID NO:1), mP6 (nt 1500-1800 of SEQ ID NO:1), mP7 (nt 1800-2100 of SEQ ID NO:1), mP8 (nt 2100-2400 of SEQ ID NO:1), mP9 (nt 2400-2700 of SEQ ID NO:1), and mP10 (nt 2700-3100 of SEQ ID NO:1). The fragments P1 and mP1 were prepared by primers pRS313/1/F of SEQ ID NO:5 and pRS313/300-hRL/R of SEQ ID NO:6. P2 and mP2 were prepared by primers pRS313/301/F of SEQ ID NO:7 and pRS313/600-hRL/R of SEQ ID NO:8. P3 and mP3 were prepared by primers pRS313/601/F of SEQ ID NO:9 and pRS313/900-hRL/R of SEQ ID NO:10. P4 and mP4 were prepared by primers pRS313/901/F of SEQ ID NO:11 and pRS313/1200-hRL/R of SEQ ID NO:12. P5 and mP5 were prepared by primers pRS313/1201/F of SEQ ID NO:13 and pRS313/1500-hRL/R of SEQ ID NO:14. P6 and mP6 were prepared by primers pRS313/1501/F of SEQ ID NO:15 and pRS313/1800-hRL/R of SEQ ID NO:16. P7 and mP7 were prepared by primers pRS313/1801/F of SEQ ID NO:17 and pRS313/2100-hRL/R of SEQ ID NO:18. P8 and mP8 were prepared by primers pRS313/2101/F of SEQ ID NO:19 and pRS313/2400-hRL/R of SEQ ID NO:20. P9 and mP9 were prepared by primers pRS313/2401/F of SEQ ID NO:21 and pRS313/2700-hRL/R of SEQ ID NO:22. P10 and mP10 were prepared by primers pRS313/2701/F of SEQ ID NO:23 and pRS313/3000-hRL/R of SEQ ID NO:24.

The wild-type fragments were amplified from viral RNA by RT-PCR, and the mutant fragments were amplified from the full length infectious clone pRS/DEN2, which is stable and amplified efficiently in bacteria. Fragments containing renilla luciferase genes were designated as HRL and cHRL. HRL was fused under the control of fragments P1, mP1, P2, mP2, P3, mP3, P4, mP4, P5, mP5, P6, mP6, P7, mP7, P8, mP8, P9, mP9, P10, and mP10. cHRL was used to make control plasmid having no fragments originated from the upstream sequence of DEN2. HRL was prepared by primers hRL/F of SEQ ID NO:25 and pRS313/hRL/R of SEQ ID NO:27. cHRL was prepared by primers pRS313/hRL/F of SEQ ID NO:26 and pRS313/hRL/R of SEQ ID NO:27. Both HRL and cHRL were amplified from the template pGL4.7-hRL (Promega, Madison, USA).

In order to make the reporter constructs pP1, pP2, pP3, pP4, pP5, pP6, pP7, pP8, pP9, pP10, pmP1, pmP2, pmP3, pmP4, pmP5, pmP6, pmP7, pmP8, pmP9, and pmP10, the fragments P1, P2, P3, P4, P5, P6, P7, P8, P9, P10, mP1, mP2, mP3, mP4, mP5, mP6, mP7, mP8, mP9, and mP10 were co-transformed respectively with HRL fragments as well as pRS313 shuttle vectors linearized by SacI into yeast strain NMY32. The yeast colonies were selected based on the presence of His⁺. The purified plasmids were re-transformed into E. coli of STBL2 strain for amplification. Control plasmid pCTL was constructed by co-transforming cHRL with pRS313 shuttle vector linearized by SacI into yeast strain NMY32.

Luciferase Activity Assay

Luciferase activity was measured by following manufacturer's instruction (Promega, Madison, USA). In brief, the reporter constructs were transformed into E. coli of STBL2 strain at the day before analysis. On the second day, three independent colonies were selected from each plate and inoculated in 3 ml Luria Broth containing 50 μg/ml ampicillin. When the O.D. 600 reached 0.6 at several hours later, 50 μl of bacteria was mixed with 40 μl water, 10 μl of 1 M K₂HPO₄ (pH 7.8), and 20 mM EDTA in a tube. The mixture was freeze-thawed once by placing the tube in liquid nitrogen followed by incubating in water bath at the room temperature. 300 μl of lysis mix (1× Cell Culture Lysis Reagent, 1.25 mg/ml lysozyme, and 2.5 mg/ml BSA) was added into cells, and the cells were incubated for 10 minutes at room temperature. 50 μl of Renilla luciferase assay reagent was mixed with 10 μl of cell lysate before measuring the activity. The measurement was performed with an one second delay followed by a ten second measurement read for luciferase activity.

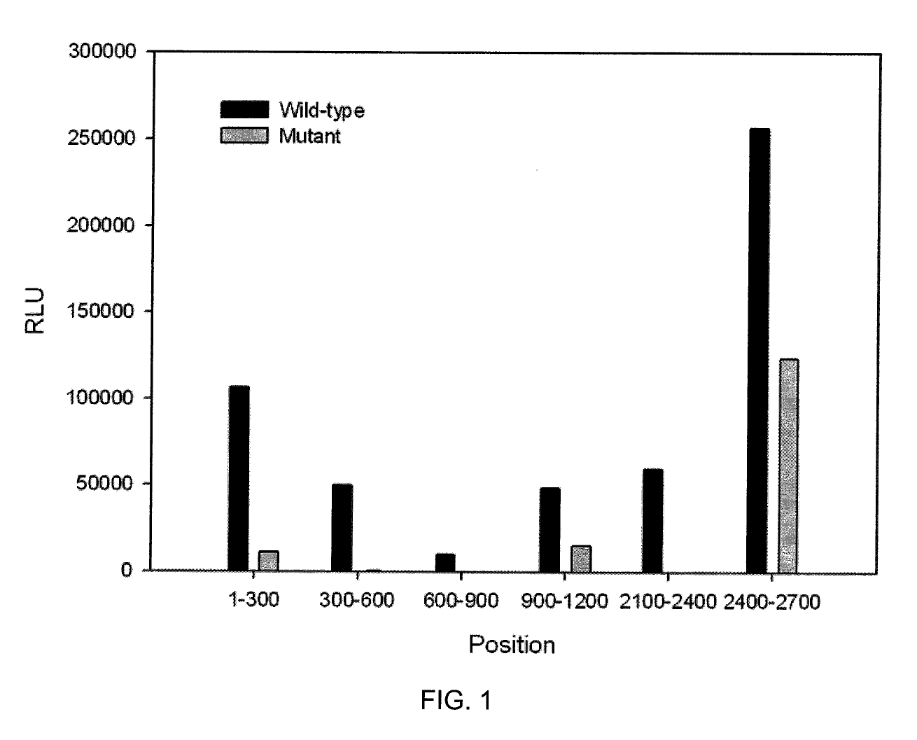

Referring to FIG. 1, the relative luciferase activities (RLU) expressed by the bacterial strains carrying the DNA fragments from wild-type and mutant DEN2 are provided. Expression of the reporter luciferase gene was observed in E. coli carrying a reporter construct of a wild-type prokaryotic promoter region operably linked to the reporter gene. The level of gene expression varied with different prokaryotic promoter regions tested, indicating that cryptic gene expression varies with the prokaryotic promoter regions tested. The mutant promoter regions tested resulted in non-expression or significant reduction in expression of the reporter gene as compared to the corresponding wild-type promoter regions. This indicated that the mutant promoter regions have no promoter activity or significantly reduced promoter activity as compared to that of the wild-type. The strongest expression of the reporter gene was found with the construct containing the nt 2400-2700 region of SEQ ID NO:1 with both the wild-type and mutant constructs.

Several prokaryotic promoter sequences in the DEN2 and JEV genome sequences were predicted based on the score of promoter activity using the Neural network promoter prediction program (http://www.fruitfly.org/seq_tools/promoter.html) from Berkley Drosophila Genome Project. Nine prokaryotic promoter regions within the core-PrM-E-NS1 region of DEN2 and three prokaryotic promoter regions within the core-PrM-E-NS1 region of JEV genomes were respectively selected according to scores of prokaryotic promoter activity (Table 1).

TABLE 1

| | | | |
|---|---|---|---|
| DEN2 | | | |
| prokaryotic | SEQ | | Promoter |
| promoter | ID | a segment of prokaryotic promoter region including | prediction |
| regions | NO: | mutations at specific nucleotide (nt) sites of DEN2 genome | score |

| | | | | |
|---|---|---|---|---|
| nt 160-205 | WT | 85 | 181 . . . ctgacAaagAgAttctcactt . . . 201 | 0.93 |
| | MT | 86 | 181 . . . ctgacGaagCgGttctcactt . . . 201 | n.d. |
| nt 198-243 | WT | 87 | 220 . . . ggaccaTtAaaActgttcatg . . . 241 | 0.95 |
| | MT | 88 | 220 . . . ggaccaCtGaaGctgttcatg . . . 241 | n.d. |
| nt 376-421 | WT | 89 | 397 . . . actgcaggcAtgatcattatg . . . 417 | 0.94 |
| | MT | 90 | 397 . . . actgcaggcCtgatcattatg . . . 417 | n.d. |
| nt 633-678 | WT | 91 | 652 . . . tccacatgggtAacttatggg . . . 672 | 0.97 |
| | MT | 92 | 652 . . . tccacatgggtGacttatggg . . . 672 | n.d. |
| nt 1059-1104 | WT | 93 | 1072 . . . ataGaaacagaagccaaacaaCctgccacTcta . . . 1104 | 0.95 |
| | MT | 94 | 1072 . . . ataAaaacagaagccaaacaaTctgccacCcta . . . 1104 | n.d. |
| nt 2104-2182 | WT | 95 | 2125 . . . tctatcggcaAaatgcttgag . . . 2145 | 0.98 |
| | MT | 96 | 2125 . . . tctatcggcaGaatgcttgag . . . 2145 | n.d. |
| nt 2582-2627 | WT | 97 | 2602 . . . acaagactggAaaatctgatg . . . 2622 | 0.96 |
| | MT | 98 | 2602 . . . acaagactggGaaatctgatg . . . 2622 | n.d. |
| nt 2615-2660 | WT | 99 | 2635 . . . acaccagaATtgaaTcacatt . . . 2655 | 1.00 |
| | MT | 100 | 2635 . . . acaccagaGCtgaaCcacatt . . . 2655 | n.d. |

| | | |
|---|---|---|
| JEV | | |
| prokaryotic | | Promoter |
| promoter | a segment of prokaryotic promoter region including | prediction |
| regions | mutations at specific nucleotide (nt) sites of JEV genome | score |

| | | | | |
|---|---|---|---|---|
| nt 60-105 | WT | 101 | 82 . . . aacggaagAtaaccatga . . . 99 | 0.94 |
| | MT | 102 | 82 . . . aacggaagCtaaccatga . . . 99 | n.d. |
| nt 72-117 | WT | 103 | 96 . . . atgacTaaAaaAccagga . . . 113 | 1.00 |
| | MT | 104 | 96 . . . atgacGaaGaaGccagga . . . 113 | n.d. |
| nt 1352-1397 | WT | 105 | 1353 . . . atTgggagaacaatccag . . . 1370 | 0.94 |
| | MT | 106 | 1353 . . . atCgggagaacaatccag . . . 1370 | n.d. |

WT: wild type; MT: mutant type; n.d.: non-detectable

By sequence analysis, the segments of prokaryotic promoter region in the mutant were found to include mutations in the prokaryotic promoter regions of DEN2 or JEV genome. The prokaryotic promoter activity of DEN2 virus was abolished (promoter activity was non-detectable) in the mutant having silent mutations in a segment of prokaryotic promoter region ranging from nt 181-201 of SEQ ID NO:1. For example, the silent mutations can include a substitution of G to A at nt 186, a substitution of C to A at nt 190 and a substitution of G to A at nt 192. Other silent mutations in the DEN2 genome can include, but are not limited to, nucleotide changes in the segments of prokaryotic promoter regions ranging from nt 220-241, nt 397-417, nt 652-672, nt 1072-1174, nt 2125-2145, nt 2602-2622 and nt 2635-2655 of SEQ ID NO:1 Also, the prokaryotic promoter activity of JEV virus was abolished when there were silent mutations in the segments of prokaryotic promoter regions ranging from nt 82-99, nt 96-113 and nt 1353-1370 of SEQ ID NO:2.

### EXAMPLE 3

### Construction of Full-Length DEN2 Infectious cDNA in Yeast and E. coli

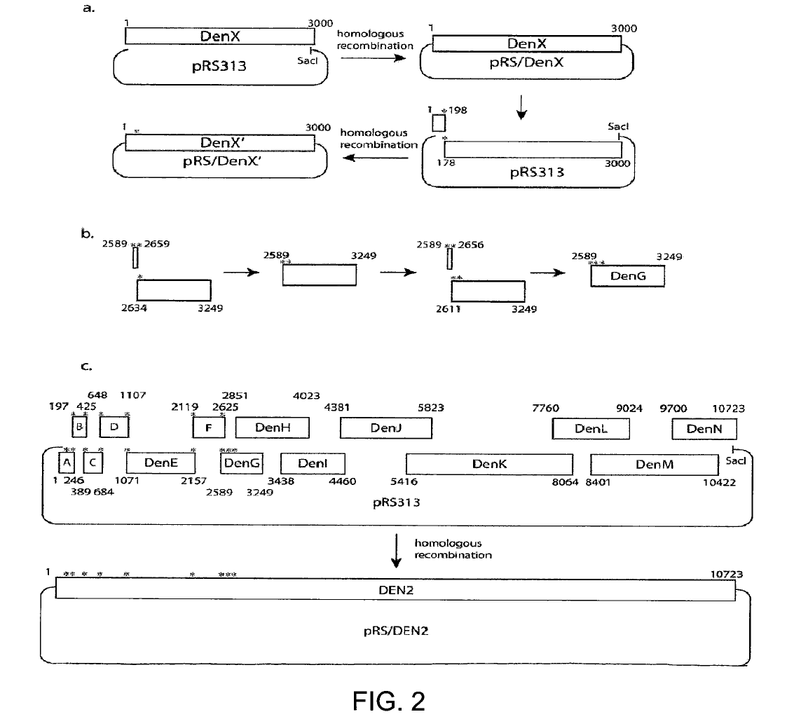

In the construction of the full-length DEN2 infectious cDNA, 14 DEN2 cDNA fragments DenA, DenB, DenC, DenD, DenE, DenF, DenG, DenH, DenI, DenJ, DenK, DenL, DenM, and DenN were assembled into full-length DEN2 cDNA in a pRS313 shuttle vector as shown in FIGS. 2a through 2c. The fragment DenA contained one bacteriophage SP6 RNA polymerase promoter sequence upstream of the 5′ end of the DEN2 genome. The fragment DenA was prepared by PCR from a plasmid pRS/DenX′, which already harbored silent mutations at nt 186, 190, and 192, with the primers pRS313-F of SEQ ID NO:28 and D2/QCM198M/R of SEQ ID NO:29.

In order to construct the pRS/DenX′, a fragment DenX was first synthesized from DEN2 viral RNA by RT-PCR with the corresponding primers D2/1-2999/F of SEQ ID NO:30 and D2/1-2999/R of SEQ ID NO:31. The D2/1-2999/F primer of SEQ ID NO: 30 was designed as a 18 mer SP6 promoter sequence at the 5′ end of dengue genome sequence. A 42 base pair (bp) homologous sequence was further added to 5′ end of the fragment DenX by PCR to re-amplify the fragment DenX containing 42 bp homologous sequence at the termini of linearized pRS313 with the corresponding primers RS/D2/1-2999/F of SEQ ID NO:32 and D2/1-2999/R of SEQ ID NO:31. Four hundred nanograms of the fragment DenX was cloned into pRS313 vector by co-transformation with 100 ng linearized pRS313 containing Sac I site into competent yeast cells of NMY32 strain (DualSystem Biotech, Zurich, Switzerland) to generate a recombinant plasmid pRS/DenX. The pRS/DenX plasmids were then purified from the yeast cells, followed by amplification in E. coli of C41 (DE3) strain (Lucigen, Middleton, Wis.).

Referring to FIG. 2a, a fragment DenX′ was prepared by introducing silent mutations at nt 186, 190, and 192 into the fragment DenX. The silent mutations were placed inside the core region within the fragment DenX by PCR-based site-directed mutagenesis, with the corresponding primers D2QCM160/F of SEQ ID NO:33 and D2QCM160/R of SEQ ID NO:34, and pRS/DenX as a template. As a result, the fragment DenX′ (nt 1 to 2999) incorporating the silent mutations was produced. 400 ng DenX′ fragment was then co-transformed with 100 ng linearized pRS313 containing Sac I site into the yeast cells of NMY32 strain which grew on solid medium lacking histidine (dropout medium). The yeast cells of NMY32 strain containing the fragment DenX′ were amplified in YEPD medium and harvested for the purification of pRS/DenX′ plasmid. Next, the pRS/DenX′ plasmid purified from the yeast cells of NMY32 strain were re-transformed into E. coli of STBL2 strain and purified.

The fragment DenB was prepared by primers D2H1/198M/F of SEQ ID NO:35 and

D2H/376M/R of SEQ ID NO:36. DenC was prepared by primers D2H/376M/F of SEQ ID NO:37 and D2H/633M/R of SEQ ID NO:38. DenD was prepared by primers D2H/633M/F of SEQ ID NO:39 and D2H/1059M/R of SEQ ID NO:40. DenE was prepared by primers D2H/1059M/F of SEQ ID NO:41 and D2H/MuK2134R/R of SEQ ID NO:42. DenF was prepared by primers D2H/MuK2134R/F of SEQ ID NO:43 and D2H/2582M/R of SEQ ID NO:44. DenG was prepared by primers D2H/2582M/F of SEQ ID NO:45 and D2/H33226/R of SEQ ID NO:46. DenH was prepared by primers D2/2850 of SEQ ID NO:47 and D2/4000/R of SEQ ID NO:48. DenI was prepared by primers PACI/3453 of SEQ ID NO:49 and D2H/4440R of SEQ ID NO:50. DenJ was prepared by primers D2H/4400 of SEQ ID NO:51 and D2/5800/R of SEQ ID NO:52. DenK was prepared by primers D2/Xh5413 of SEQ ID NO:53 and D2/8047/R of SEQ ID NO:54. DenL was prepared by primers PRS313/D2NGC/7760F of SEQ ID NO:55 and D2/9001/R of SEQ ID NO:56. DenM was prepared by primers D2/8401 of SEQ ID NO:57 and D2/10399/R of SEQ ID NO:58. DenN was prepared by primers D2/9700 of SEQ ID NO:59 and PRS313/D2NGC/XbaI-10724R of SEQ ID NO:3.

Silent mutations at nt 226, 228, and 231 of DEN2 were incorporated by primers D2/QCM198M/R of SEQ ID NO:29 and D2H/198M/F of SEQ ID NO:35. A silent mutation at nt 406 of DEN2 was incorporated by primers D2H/376M/R of SEQ ID NO:36 and D2H/376M/F of SEQ ID NO:37. A silent mutation at nt 663 of DEN2 was incorporated by primers D2H/663M/R of SEQ ID NO:38 and D2H/663M/F of SEQ ID NO:39. Silent mutations at nt 1093 and 1101 of DEN2 were incorporated by primers D2H/1059M of SEQ ID NO:40 and D2H/1059M/F of SEQ ID NO:41. A mutation at nt 2135 of DEN2 that replaced amino acid lysine with arginine was incorporated by primers D2H/MuK2134R/R of SEQ ID NO:42 and D2H/MuK2134R/F of SEQ ID NO:43. A silent mutation at nt 2612 of DEN2 was incorporated by primers D2H/2582M/R of SEQ ID NO:44 and D2H/2582M/F of SEQ ID NO:45. Silent mutations at nt 2631 and 2634 of DEN2 were incorporated by primers PLH/8M/m2604/R of SEQ ID NO:60 and PLH/8M/m2604/F of SEQ ID NO:61.

Referring to FIG. 2b, the DenG fragment which contained silent mutations at nt 2643, 2644, and 2649 of DEN2 was synthesized by PCR with the corresponding mutagenic primers, D2QCM/2615F of SEQ ID NO:62 and D2QCM/2615R of SEQ ID NO:63, as well as the primers D2H/2582M/F of SEQ ID NO:45 and D2/H33226/R of SEQ ID NO:46. All the fragments except DenA were synthesized from viral RNA by RT-PCR.

Referring to FIG. 2c, the fragments were co-transformed into yeast cells of NMY32 strain with the shuttle vector pRS313 linearized by SacI to accomplish full-length DEN2 infectious cDNA constructs. The yeast colonies were selected based on the presence of His⁺. The purified plasmids were re-transformed into E. coli of C41 (DE3) strain for amplification and subjected to sequencing analysis performed on ABI genetic analyzer.

### EXAMPLE 4

### Construction of Full-Length JEV Infectious cDNA in Yeast and E. coli

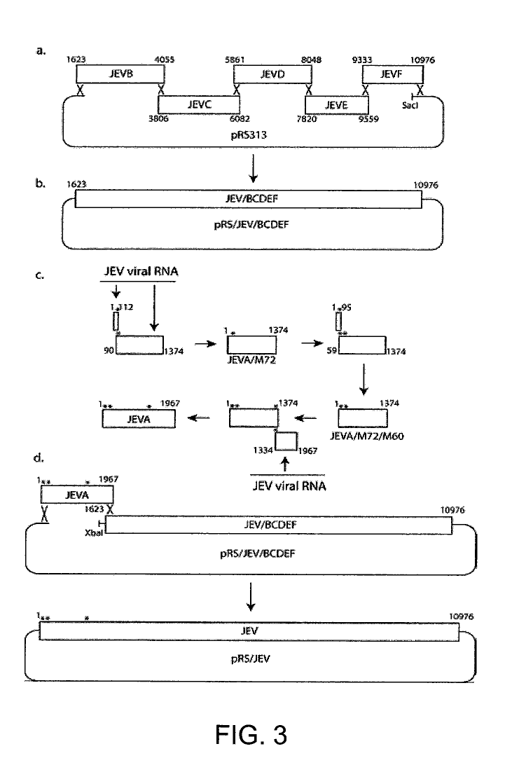

Similar strategy was used to construct the full-length JEV infectious cDNA. Five JEV cDNA JEVB (nt 1623-4055 of JEV), JEVC (nt 3806-6082 of JEV), JEVD (nt 5861-8048 of JEV), JEVE (nt 7820-9559 of JEV), and JEVF (nt 9333-10976 of JEV) were first assembled into pRS/JEV/BCDE in a pRS313 shuttle vector as shown in FIGS. 3a and 3b. Fragment JEVB was prepared by primers RU-SP6-JEV1623 of SEQ ID NO:64 and JEV-4055R of SEQ ID NO:65. JEVC was prepared by primers JEV-3806 of SEQ ID NO:66 and JEV-6082R of SEQ ID NO:67. JEVD was prepared by primers JEV-5861 of SEQ ID NO:68 and JEV-8048R of SEQ ID NO:69. JEVE was prepared by primers JEV-7820 of SEQ ID NO:70 and JEV-9559R of SEQ ID NO:71. Fragment JEVF was prepared by primers JEV-9333 of SEQ ID NO:72 and JEV-10976-BsrGI of SEQ ID NO:73. All these fragments were amplified from viral RNA by RT-PCR and co-transformed with pRS313 linearized with SacI into yeast cells of NMY32 strain. The yeast colonies were selected based on the presence of His⁺. The pRS/JEV/BCDE plasmid was purified from yeast cells of NMY32 strain and re-transformed into E. coli of C41 (DE3) strain to amplify enough amount for DNA manipulation and sequence analysis on ABI genetic analyzer.

The fragment JEVA contained one SP6 RNA polymerase promoter sequence upstream of the 5′ end of the JEV genome and several silent mutations. Silent mutations at nt 101, 104, and 107 of JEV on JEVA were first introduced by PCR-based mutagenesis with mutagenic primers JEV/RP9/QCM72/R of SEQ ID NO:74 and JEV/RP9/QCM72/F of SEQ ID NO:75, as well as primers pRS313/JEVRP9/SacI+SP6-long of SEQ ID NO:76 and JEV-1352M-R of SEQ ID NO:77. As shown in FIG. 3c, the resulted JEVA/M72 fragment was used as template in the second round mutagenesis with primers JV60M-1R of SEQ ID NO:78 and JV60M-1 of SEQ ID NO:79 to add a silent mutations at nt 90 of JEV to make the fragment JEVA/M72/M60. Next, another silent mutation at nt 1355 of JEV was added to JEVA/M72/M60 by PCR-based mutagenesis using primers pRS313/JEVRP9/SacI+SP6-long of SEQ ID NO:76 and JEV-1967R of SEQ ID NO:80, as well as JEVA/M72/M60 and the PCR product of primers JEV-1352M of SEQ ID NO:81 and JEV-1967R of SEQ ID NO:80 as template.

Finally, the JEVA fragment was co-transformed into yeast cells of NMY32 strain with pRS/JEV/BCDE linearized by XhoI to generate full length JEV infectious cDNA through homologous recombination as shown in FIG. 3d. The yeast colonies were selected based on the presence of His⁺. The pRS/JEV plasmid was purified from yeast cells of NMY32 strain and re-transformed into E. coli of C41 (DE3) strain to amplify enough amount for DNA manipulation and sequence analysis on ABI genetic analyzer.

### EXAMPLE 5

### In Vitro Transcription and Transfection of DEN2 or JEV Viral RNA

Each of the four pRS/DEN2 or pRS/JEV plasmids containing full-length DEN2 or JEV cDNA constructed, respectively, according to examples 3 or 4 was linearized with XbaI or BsrGI, treated with Mung Bean Nuclease (New England Biolabs, Massachusetts, USA), extracted with phenol-chlorofom followed by ethanol precipitation. For in vitro RNA synthesis, the transcription mixture contained 2 μg of linearized DNA; 5 mM each ATP, CTP, and UTP; 3 mM GTP; 4 mM cap analog m7G(5′)ppp(5′)G ; 2 μl of SP6 enzyme mix; and 1×SP6 reaction buffer in a volume of 20 μl (Ambion, Austin, Tex.). The reaction mixture was incubated at 37° C. for 2 hours. One micro-liter of the reaction mixture was loaded on agarose gel electrophoresis. The typical yield of RNA was approximately 15 μg.

Transfection is carried out by incubating about 5 μg of in-vitro transcribed full length DEN2 or JEV viral RNA with 20 μl of Lipofectin (Invitrogen, Carlsbad, Calif.) in 1 ml of Opti-MEM medium before transferring Lipofectin-RNA mixture to twice-washed 75% confluent BHK21 cells in 35 mm dishes at 37° C. After 5 hours of incubation, the Lipofectin-RNA mixture is removed and fed with MEM maintenance media containing 2% fetal bovine serum for three days. Virus particles are harvested from the supernatant of transfected BHK21 cells 3 days post transfection and amplified in C6/36 cells for two passages and the amplified virus particles are applied to native BHK21 cells to determine whether they cause cytopathic effect (CPE) in BHK21 cells or not. In addition, the plaque assay is used to determine the titer of the amplified virus particles. Then, virus growth curve is measured and compared between transcript-derived viruses and parental virus stocks. The replication kinetics of transcript-derived viruses also provide one way to show the infectivity of infectious cDNA clone.

The purified plasmids from 4 colonies were examined by restriction enzyme digestion to verify the presence of the modified cDNA. The plasmids from 4 colonies had correct pattern of restriction enzyme digestion and the yield is about 0.8 μg/ml for mutated DEN PL046 clone and 0.7 μg/ml for the mutated JEV RP9 clone as listed in Table 2 below.

TABLE 2

| Full length infectious cDNA clone | colonies of E. coli. | DNA yield |
|---|---|---|
| Wild type DEN2 PL046 | 0/8 | n.d.* |
| Mutated DEN2 PL046 (8M) | 4/4 | ~0.8 μg/ml LB |
| Wild-type JEV RP9 | unavailable† | n.d.* |
| Mutated JEV RP9 (TM) | 4/4 | ~0.7 μg/ml LB |

*n.d. no data available
†Partial wild type JEV RP9 DNA sequence is unable to obtain in E. coli because JEV cDNA is toxic to E. coli.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method for amplifying a functional flavivirus cDNA in a prokaryotic cell, comprising:
(a) constructing a modified flavivirus cDNA by introducing a silent mutation into a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;
(b) introducing the modified flavivirus cDNA into the prokaryotic cell; and
(c) amplifying the functional flavivirus cDNA by replication of the modified flavivirus cDNA in the prokaryotic cell.

2. The method according to claim 1, wherein the flavivirus is selected from the group consisting of a dengue virus (DEN), Japanese encephalitis virus (JEV), West Nile virus (WNV), yellow fever virus (YFV), and tick-borne encephalitis virus (TBE).

3. The method according to claim 1, wherein the flavivirus cDNA comprises SEQ ID NO:1 or SEQ ID NO:2.

4. The method according to claim 3, wherein the prokaryotic promoter region is selected from the group consisting of nt 160-205, 198-243, 376-421, 633-678, 1059-1104, 2104-2182, 2582-2627 and 2615-2660 of SEQ ID NO:1.

5. The method according to claim 4, wherein the silent mutation is introduced to SEQ ID NO:1 at a position selected from group consisting of nt 186, 190, 192, 226, 228, 231, 406, 663, 1093, 1101, 2135, 2612, 2643, 2644 and 2649 of SEQ ID NO:1.

6. The method according to claim 3, wherein the prokaryotic promoter region is selected from the group consisting nt 60-105, 72-117 and 1352-1397 of SEQ ID NO:2.

7. The method according to claim 6, wherein the silent mutation is introduced into SEQ ID NO:2 at a position selected from group consisting of nt 90, 101, 104, 107 and 1355 of SEQ ID NO:2.

8. The method according to claim 1, wherein the prokaryotic cell is an Escherichia coli cell.

9. The method according to claim 1, wherein the silent mutation is selected from the group consisting of an A to C substitution, A to G substitution, C to T substitution, T to C substitution and T to G substitution.

10. The method according to claim 1, wherein the modified flavivirus cDNA comprises two or more silent mutations in one or more prokaryotic promoter regions.

11. The method according to claim 1, further comprising identifying the prokaryotic promoter region based on sequence analyses of the flavivirus cDNA.

12. An isolated nucleic acid molecule selected from the group consisting of:
(i) a modified flavivirus cDNA comprising a silent mutation in a prokaryotic promoter region within a flavivirus cDNA, wherein the silent mutation decreases or abolishes the promoter activity from the prokaryotic promoter region without resulting in a change to the amino acid sequence encoded by the modified flavivirus cDNA as compared to that encoded by the flavivirus cDNA;
(ii) a complement of the modified flavivirus cDNA; and
(iii) an RNA transcript of the modified flavivirus cDNA.

13. The isolated nucleic acid molecule of claim 12, wherein the flavivirus cDNA comprises SEQ ID NO:1 or SEQ ID NO:2.

14. The isolated nucleic acid molecule of claim 13, wherein the prokaryotic promoter region is selected from the group consisting of nt 160-205, 198-243, 376-421, 633-678, 1059-1104, 2104-2182, 2582-2627 and 2615-2660 of SEQ ID NO:1.

15. The isolated nucleic acid molecule of claim 14, wherein the silent mutation is at a position selected from group consisting of nt 186, 190, 192, 226, 228, 231, 406, 663, 1093, 1101, 2135, 2612, 2643, 2644 and 2649 of SEQ ID NO:1.

16. The isolated nucleic acid molecule of claim 13, wherein the prokaryotic promoter region is selected from the group consisting of nt 60-105, 72-117 and 1352-1397 of SEQ ID NO:2.

17. The isolated nucleic acid molecule of claim 16, wherein the silent mutation is at a position selected from group consisting of nt 90, 101, 104, 107 and 1355 of SEQ ID NO:2.

18. The isolated nucleic acid molecule of claim 12, wherein the RNA transcript is produced from an in vitro transcription system.

19. The isolated nucleic acid molecule of claim 12, wherein the silent mutation is selected from the group consisting of an A to C substitution, A to G substitution, C to T substitution, T to C substitution and T to G substitution.

20. A vector comprising the modified flavivirus cDNA or the complement thereof according to claim 12.

21. A prokaryotic cell comprising the vector according to claim 20.

22. A flavivirus produced by a host cell transfected with the RNA transcript according to claim 12.

23. The flavivirus according to claim 22 being selected from the group consisting of a dengue virus (DEN), Japanese encephalitis virus (JEV), West Nile virus (WNV), yellow fever virus (YFV), and tick-borne encephalitis virus (TBE).

24. The flavivirus according to claim 22 being a DEN, wherein the DEN has a cDNA comprising SEQ ID NO:1 and at least one silent mutation at a prokaryotic promoter region selected from the group consisting of nt 160-205, 198-243, 376-421, 633-678, 1059-1104, 2104-2182, 2582-2627 and 2615-2660 of SEQ ID NO:1.

25. The flavivirus according to claim 22 being a JEV, wherein the JEV has a cDNA comprising SEQ ID NO:2 and at least one silent mutation at a prokaryotic promoter region selected from the group consisting of nt 60-105, 72-117 and 1352-1397 of SEQ ID NO:2.


SEQUENCE LISTING (106 sequences)

SEQ ID NO: 1 - DNA, 10722 bp, Dengue virus type 2
agttgttagtctacgtggaccgacaaagacagattctttgagggagctaagctcaacgtagttctaacagttttttaattagagagcagatctctgatgaataaccaacgaaaaaaggcgagaaatacgcctttcaatatgctgaaacgcgagagaaaccgcgtgtcgactgtacaacagctgacaaagagattctcacttggaatgctgcagggacgaggaccattaaaactgttcatggccctggtggcgttccttcgtttcctaacaatcccaccaacagcagggatactgaagagatggggaacaattaaaaaatcaaaagccattaatgttttgagagggttcaggaaagagattggaaggatgctgaacatcttgaacaggagacgcagaactgcaggcatgatcattatgctgattccaacagtgatggcgttccatttaaccacacgtaacggagaaccacacatgatcgtcagtagacaagagaaagggaaaagtcttctgtttaaaacaggggatggtgtgaacatgtgtaccctcatggccatggaccttggtgaattgtgtgaagatacaatcacgtacaagtgtccttttctcaggcagaatgaaccagaagacatagattgttggtgcaactctacgtccacatgggtaacttatgggacgtgtaccaccacaggagaacacagaagagaaaaaagatcagtggcactcgttccacatgtgggaatgggactggagacacgaactgaaacatggatgtcatcagaaggggcctggaaacatgcccagagaattgaaacttggatcttgagacatccaggctttaccataatggcagcaatcctggcatacaccataggaacgacacatttccaaagagccctgattttcatcttactgacagctgtcgctccttcaatgacaatgcgttgcataggaatatcaaatagagactttgtagaaggggtttcaggaggaagctgggttgacatagtcttagaacatggaagctgtgtgacgacgatggcaaaaaacaaaccaacattggattttgaactgatagaaacagaagccaaacaacctgccactctaaggaagtactgtatagaggcaaagctgaccaacacaacaacagaatctcgctgcccaacacaaggagaacccagcctaaatgaagagcaggacaaaaggttcgtctgcaaacactccatggtggacagaggatggggaaatggatgtggattatttggaaaaggaggcattgtgatctgtgctatgttcacatgcaaaaagagcatgaaaggaaaagtcgtgcaaccagaaaacttggaatacaccattgtgataacacctcactcaggggaagagcatgcattcggaaatgacacaggaaaacatggcaaggaaatcaaaataacaccacagagttccatcacagaagcaaagttgacaggctatggcactgtcacgatggagtgctctccgagaacgggcctcgacttcaatgagatggtgttgctgcaaatggaagataaagcttggctggtgcacaggcaatggttcctagacctgccgttgccatggctgcccggagcggacacacaaggatcaaattggatacagaaagagacattggtcactttcaaaaatcctcatgcgatgaaacaggatgttgttgttttgggatcccaagaaggggccatgcacacagcactcacaggggccacagaaatccagatgtcatcaggaaacttactgttcacaggacatctcaggtgcaggctgaggatggacaaactacagctcaaaggaatgtcatactctatgtgcacaggaaagtttaaagttgtgaaggaaatagcagaaacacaacatggagcaatagttatcagagtacaatatgaaggggacggttctccatgtaagatcccttttgagataatggatttggaaaaaagacatgttttaggtcgcctgattacagtcaacccaatcgtaacagaaaaagatagcccagtcaacatagaagcagaacctccattcggagacagctacatcatcataggagtagagccgggacaattgaagctcaactggtttaagaaaggaagttctatcggcaaaatgcttgagacaacaatgaggggagcgaagagaatggccattttaggtgacacagcttgggattttggatccctgggaggagtgtttacatctataggaaaggctctccaccaagttttcggagcaatctatggggctgccttcagtggggtctcatggactatgaaaatcctcataggagtcattatcacatggataggaatgaattcacgcagcacctcactgtctgtgtcactagtattggtgggagtcgtgacgctgtatttgggagttatggtgcaggccgatagtggttgcgttgtgagctggaaaaacaaagaactgaagtgtggcagtgggattttcatcacagacaacgtgcacacatggacagaacaatacaagttccaaccagaatccccttcaaaactagcttcagctatccagaaagctcatgaagagggcatttgtggaatccgctcagtaacaagactggaaaatctgatgtggaaacaaataacaccagaattgaatcacattctatcagaaaatgaggtgaagttgactattatgacaggagacatcgaaggaatcatgcaggcaggaaaacgatctctgcggccccagcccactgagctgaagtattcatggaaaacatggggcaaagcgaaaatgctctctacagagtctcataaccagacctttctcattgatggccccgaaacagcagaatgccccaacacaaacagagcttggaattcgctggaagttgaagactatggctttggagtattcaccaccaatatatggctaaggttgagagaaaagcaggatgtattctgcgactcaaaactcatgtcagcggccataaaagacaacagagccgtccatgccgatatgggttattggatagaaagtgcactcaacgacacatggaagatagagaaagcctctttcatcgaagttaaaagctgccactggccaatgtcacacaccctctggagtaatgaagtgctagaaagtgagatgataattccaaagaatttcgctggaccagtgtcacaacacaactacagaccaggctaccatacacaaacagcaggaccatggcatctaggtaagcttgagatggactttgatttctgcgaaggaaccacagtggtggtgactgaggactgtggaaatagaggaccctctttaagaacaactactgcctctggaaaactcatagcagaatggtgctgccgatcttgcacattaccaccgctaagatacagaggtgaggacggatgctggtacgggatggaaatcagaccattgaaagagaaagaagagaatttaattaactccttggtcacagccggacatgggcagattgacaacttttcactaggagtcttgggaatggcattgttcctggaagaaatgctcaggacccgagtaggaacgaaacatgcaatactactagttgcagtttcttttgtgacattgatcacagggaacatgtcctttagagacctgggaagagtgatggttatggtgggcgctactatgacggatgacataggtatgggtgtgacttatcttgccctactagcagccttcaaagtcagaccgacttttgcagctggactactcttgagaaagttgacctccaaggaactgatgatgactaccataggaatcgcactcctctcccagagcaccataccagagaccattcttgaactgactgacgcgttagccttgggcatgatggtccttaaaatggtgagaaaaatggaaaagtatcaattggcagtgactatcatggctatcttgtgcgtcccaaatgcagtgatattacaaaacgcatggaaagtgagttgcacaatattggcagtggtgtccgtttccccactgttcttaacatcctcacagcagaaagcggattggataccattagcattgacgatcaagggtctcaatccaacagctatttttctaacaaccctttcaagaactaacaagaaaaggagctggccactaaatgaggctatcatggcagtcgggatggtgagcattttggccagttcactcctaaagaatgacattcccatgacaggaccattagtggctggagggctcctcactgtgtgctacgtgctcactggacgatcggccgatttggaactggagagagccgccgatgtcaaatgggaagatcaggcagagatatcaggaagcagtccaatcctatcaataacaatatcagaagatggtagcatgtcgataaaaaacgaagagggagaacaaacactgaccatactcattagaacaggattgctggtgatctcaggactttttcctgtatcactaccaatcacggcagcagcatggtacctgtgggaagtgaaaaaacaacgggctggagtattgtgggatgtcccttcacccccacccgtaggaaaggctgaactggaagatggagcctatagaatcaagcaaaaagggattcttggatattcccagatcggagccggagtttacaaagaaggaacattccatacaatgtggcatgtcacacgcggcgctgttctaatgcataaaggaaagaggattgaaccatcatgggcggacgttaagaaagacctaatatcatatggaggaggctggaagctagaaggagaatggaaggaaggagaagaagtccaggtcttggcattggagccaggaaaaaatccaagagccgtccaaacaaaacctggtcttttcaaaaccaacgccggaaccataggtgccgtatctctggacttttctcctggaacctcaggatctccaatcatcgacaaaaaaggaaaagttgtgggtctttatggtaatggtgttgttacaaggagtggagcatatgtaagtgctatagcccagactgaaaaaagtattgaagacaatccagagatcgaagatgacatttttcgaaagagaaaattgaccatcatggacctccacccaggagcgggaaagacgaagagataccttccggccatagtcagagaggctataaaacggggcctgaggacattaatcctggcccccactagagtcgtggcagctgaaatggaggaagccctaagaggacttccaataagataccaaaccccagccatcagagctgagcacaccgggcgggagattgtggacctaatgtgtcatgccacattcactatgaggctgctatcaccagttagagtgccaaattacaacctgatcatcatggacgaagcccatttcacagacccagcaagtatagcggctagaggatacatctcaactcgagtagagatgggtgaggcagctgggattttcatgacagccactcctccgggaagcagagacccattccctcagagcaatgcaccaatcatggatgaagaaagagaaatccctgaacgttcgtggagttctggacatgagtgggtcacggattttaaagggaagactgtttggttcgttccaagtataaaagcaggaaatgatatagcagcttgcctgagaaaaaatggaaagaaagtgatacaactcagtaggaagacctttgattctgagtatgtcaagactagaaccaatgattgggacttcgtggtcacaactgacatttcagaaatgggtgccaacttcaaggctgagagggttatagaccccagacgctgcatgaaaccagttatactaacagatggtgaagagcgggtgatccttgcaggacctatgccagtgacccactctagtgcagcacaaagaagagggagaataggaagaaatccaaaaaatgaaaatgaccagtacatatacatgggggaacctctggaaaatgatgaagattgtgcacattggaaagaagctaaaatgctcctagataacatcaacacacctgaaggaatcattcctagcatgttcgaaccagagcgtgaaaaggtggatgccattgatggtgaataccgcttgagaggagaagcaaggaaaacctttgtggacctaatgagaagaggagacctaccagtctggttggcctacagagtggcagctgaaggcatcaactacgcagacagaaggtggtgttttgatggaattaagaacaaccaaatcttggaagaaaatgtggaggtggaaatctggacaaaagaaggggaaaggaagaaattaaaacccagatggttggatgccaggatctactctgacccactggcgctaaaggaattcaaggagtttgcagctggaagaaagtccctgaccctgagcctaatcacagaaatgggtaggcttccaactttcatgactcagaaggcaagagacgcactggacaacttagcagtgctgcacacggctgaagcaggtggaagggcgtacaatcatgctctcagtgaactgccggagaccctggagacattgcttttactgacacttctggctacagtcacgggaggaatctttttattcttgatgagcggaaggggtatagggaagatgaccctgggaatgtgctgcataatcacggctagtactctcctatggtacgcacaaatacagccacactggatagcagcttcaataatactggggttttttctcatagttttgcttattccagaaccagaaaagcagagaacaccccaagataaccaattgacctacgttgtcatagccatcctcacagtggtggccgcaaccatggcaaacgggatgggtttcctggaaaaaacgaagaaagatctcggattgggaagcattacaacccagcaacccgagagcaacatcctggacatagatctacgtcccgcatcagcatggacgctgtatgctgtggccacaacatttgtcacaccaatgttgagacacagcattgaaaattcctcagtgaacgtgtccctaacagccattgccaaccaagccacagtgttaatgggtcttgggaaaggatggccattgtcaaagatggacatcggagttccccttctcgccattggatgctactcacaagtcaaccccataactctcacagcagctcttttcttactggtagcacattatgccatcatagggccaggactccaagcaaaagcaaccagagaagctcagaaaagagcagcagcgggcatcatgaaaaacccaactgtcgatggaataacagtgattgacctagatccaataccctatgatccaaagtttgaaaagcagttgggacaagtaatgctcctagtcctctgcgtgactcaagtgttgatgatgaggactacatgggctctgtgtgaggctttaaccttagcgaccgggcctatctccacattgtgggaaggaaatccagggaggttttggaacactaccatcgcagtgtcaatggctaacatttttagagggagttacttggccggagctggacttctcttttccatcatgaagaacacaaccaacacgagaaggggaactggcaacataggagagacgcttggagagaaatggaaaagccgattgaacgcattggggaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgtcgagagaaatatggtcacaccagaagggaaagtagtggacctcggttgcggcagaggaggctggtcatactattgtgggggactaaagaatgtaagagaagtcaaaggcctaacaaaaggaggaccaggacatgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtggaaaattggttgaacaacaacactcaattttgcataaaggttctcaacccatatatgccctcagtcatagaaaaaatggaaacactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatattgacctcggaagcggaacccgcaacatcgggattgaaagtgagataccaaacctagatataattgggaaaagaatagaaaaaataaaacaagagcatgaaacatcatggcactatgaccaagaccacccatacaaaacgtgggcgtaccatggtagctatgaaacaaaacaaactggatcagcatcatccatggtcaacggagtggtcaggctgctgacaaaaccttgggacgttgtccccatggtgacacagatggcaatgacagacacgactccatttggacaacagcgcgtttttaaagaaaaagtggacacgagaacccaagaaccgaaagaaggcacaaagaaactaatgaaaatcacggcagagtggctttggaaagaactagggaagaaaaagacacccaggatgtgcaccagagaagaattcacaagaaaggtgagaagcaatgcagccttgggggccatattcactgatgagaacaagtggaagtcggcacgtgaggctgttgaagatagtaggctttgggagctggttgacaaggaaaggaatcttcatcttgaaggaaagtgtgaagcatgtgtgtacaacatgatgggaaaaagagagaagaagctaggggaattcggcaaggcaaaaggcagcagagccatatggtacatgtggcttggagcacgcttcttagagtttgaagccctaggattcttgaatgaagatcactggttctccagagagaactccttgagtggagtggaaggagaagggctgcacaagctaggttacattttaagagacgtgagcaagaaagagggaggagcaatgtatgccgatgacaccgcaggatgggacacaagaatcacactagaagacctaaaaaacgaagaaatggtaacaaaccacatggaaggagaacacaagaaactagccgaggccattttcaaattaacgtaccaaaacaaggtggtgcgtgtgcaaagaccaacaccaagaggcacagtaatggatatcatatcgagaagagaccaaagaggtagtggacaagttggtacctatggactcaatactttcaccaatatggaagcccaactaatcagacagatggagggagaaggagtcttcaaaagcattcagcacctgacagtcacagaagaaatcgccgtgcaaaactggttagcgagagtagggcgcgaaaggttatcaagaatggccatcagtggagatgattgtgttgtgaaacctttagatgacaggttcgcaagcgctttaacagctctaaatgacatgggaaaggttaggaaagacatacaacaatgggaaccttcaagaggatggaacgattggacacaagtgcccttctgttcacaccatttccatgagttaatcatgaaagacggccgcgtacttgtagttccatgcagaaaccaagatgaactgattggtagagcccgaatttcccaaggagctgggtggtctttgcgagagacggcctgtttggggaagtcctacgcccaaatgtggagcttgatgtacttccacagacgtgacctcaggctggcggctaatgctatttgctcggcagtcccatcacattgggttccaacaagtagaacaacctggtccatacacgccaaacatgaatggatgacagcggaagacatgctgacagtctggaacagggtgtggattcaagaaaacccatggatggaagacaaaactccagtggaatcatgggaggaaatcccatacttggggaaaagagaagaccaatggtgcggctcattgattgggctaacaagcagggccacctgggcaaagaacatccaaacagcaataaatcaagttagatcccttataggcaatgaggaatacacagattacatgccatccatgaaaagattcagaagagaagaggaagaggcaggagtcctgtggtagaaggcaaaactaacatgaaacaaggctagaagtcaggtcggattaagccatagtacggaaaaaactatgctacctgtgagccccgtccaaggacgttaaaagaagtcaggccattacaaatgccatagctcgagtaaactgtcagcccgtagctccacctgagaaggtgtaaaaaatctgggaggccacaaaccatggaagctgtacgcatggcgtggtggactagcggttagaggagacccctcccttacaaatcgcagcaacaatgggggcccaaggtgagatgaagcagtagtctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 2 - DNA, 10976 bp, Japanese encephalitis virus
agaagtttatctgtgtgaacttcttggcttagtatcgttgagaagaatcgagagattagtgcagtttaaacagttttttagaacggaagataaccatgactaaaaaaccaggagggcccggtaaaaaccgggctaccaatatgctgaaacgcggcctaccccgcgtattcccactagtgggagtgaagagggtagtaatgagcttgttggacggcagagggccagtacgtttcgtgctggctcttatcacgttcttcaagtttacagcattagccccgaccaaggcgcttctaggccgatggaaagcagtggaaaagagtgtagcaatgaaacatctcactagtttcaaacgagaacttggaacactcattgacgccgtgaacaagcggggcagaaagcaaaacaaaagaggaggaaatgaaggctcaatcatgtggctcgcgagcttggcagttgtcatagcttgtgcaggagccatgaagttgtcaaatttccaggggaagcttttgatgaccattaacaacacggacattgcagacgttatcgtgattcccacctcaaaaggagagaacagatgctgggtccgggcaatcgacgccggctacatgtgtgaggacactatcacgtacgaatgtcctaagcttaccatgggcaatgatccagaggatgtggattgctggtgtgacaaccaagaagtctacgtccaatatggacggtgcacgcggaccagacattccaagcgaagcaggagatccgtgtcggtccaaacacatggggagagttcactagtgaataaaaaagaggcttggctggattcaacgaaagccacacgatatctcatgaaaactgagaactggatcataaggaatcctggctatgctttcctggcggcggtacttggctggatgcttggcagtaacaacggtcaacgcgtggtattcaccatcctcctgctgctggttgctccggcttacagttttaattgtctgggaatgggcaatcgtgacttcatagaaggagccagtggagccacttgggtggacttggtgctagaaggagatagctgcttgacaattatggcaaacgacaaaccaacattggacgtccgcatgatcaacatcgaagctagccaacttgctgaggtcagaagttactgttatcatgcttcagtcactgacatctcaacggtggctcggtgccccacgactggagaagcccacaacgagaagcgagctgatagtagctatgtgtgcaaacaaggcttcactgatcgtgggtggggcaacggatgtggacttttcgggaagggaagcattgacacatgtgcaaaattctcctgcaccagtaaagcgattgggagaacaatccagccagaaaacatcaaatacgaagttggcatttttgtgcatggaaccaccacttcggaaaaccatgggaattattcagcgcaagttggggcgtcccaggcggcaaagtttacagtaacacccaatgctccttcgataaccctcaaacttggtgactacggagaagtcacactggactgtgagccaaggagtggactgaacactgaagcgttttacgtcatgaccgtggggtcaaagtcatttctggtccatagggaatggtttcatgacctcgctctcccctggacgtccccttcgagcacagcgtggagaaacagagaactcctcatggagtttgaagaggcgcacgccacaaaacagtccgttgttgctcttgggtcacaggaaggaggcctccatcaggcgttggcaggagccatcgtggtggagtactcaagctcagtgaagttaacatcaggccacctgaaatgcaggctgaaaatggacaaactggctctgaaaggcacaacctatggcatgtgcacagaaaaattctcgttcgcaaaaaatccggcggacactggtcacggaacagttgtcatcgaactctcctactctgggagtgatggcccctgcaaaattccgattgtctccgttgcgagcctcaatgacatgacccccgttgggcggctggtgacagtgaaccccttcgtcgcgacttccagtgccaattcaaaggtgctggtcgagatggaaccccccttcggagactcctacatcgtagttggaaggggagacaagcagatcaaccaccattggcacaaagctggaagcacgctgggcaaagccttttcaacaactttgaagggagctcagagactggcagcgttggatgacacagcctgggactttggctccattggaggggtcttcaactccataggaaaagccgttcaccaagtgtttggtggtgccttcagaacactctttgggggaatgtcttggatcacacaagggctaatgggtgccctactactctggatgggcgtcaacgcacgagaccgatcaattgctttggccttcttagccacaggaggtgtgctcgtgttcttagcgaccaatgtgcatgctgacactggatgtgccattgacatcacaagaaaagagatgaggtgtggaagtggcatcttcgtgcacaacgacgtggaagcctgggtggataggtataaatatttgccagaaacgcccagatccctagcgaagatcgtccacaaagcgcacaaggaaggcgtgtgcggagtcagatctgtcactagactggagcatcaaatgtgggaagccgtacgggatgaattgaacgtcctgctcaaagagaatgcagtggacctcagtgtggttgtgaacaagcccgtggggagatatcgctcagcccctaaacgcctatccatgacgcaagagaagtttgaaatgggctggaaagcatggggaaaaagcattctctttgccccggaattggctaactccacatttgtcgtagatggacctgagacaaaggaatgccctgatgagcacagagcttggaacagcatgcaaatcgaagacttcggctttggcatcacatcaacccgtgtgtggctgaagattagagaggagagcactgacgagtgtgatggagcggtcataggtacggctgtcaaaggacatgtggcagtccatagtgacttgtcgtactggattgagagtcgctacaacgacacatggaaacttgagagggcagtctttggagaggttaaatcttgcacttggccagagacacacaccctatggggagatggtgttgaggaaagtgaactcatcattccgcataccatagccggaccaaaaagcaagcacaatcggagggaagggtataagacacaaaaccagggaccttgggacgagaatggcatagtcttggactttgactattgcccagggacaaaagtcaccattacagaggattgtggcaagagaggcccttcggtcagaaccactactgacagtggaaagttgatcactgactggtgctgtcgcagttgctcccttccgcccctacgattccggacagaaaatggctgctggtacggaatggaaatcagacctgttaggcatgatgaaacaacactcgtcagatcacaggttgatgcttttaatggtgaaatggttgacccttttcagctgggccttctggtgatgtttctggccacccaggaggtccttcgcaagaggtggacggccagattgaccattcctgcggttttgggagccttacttgtgctgatgcttgggggcatcacttacactgatttggcgaggtatgtggtgctagtcgctgctgctttcgcagaggccaacagtggaggagacgtcctgcaccttgctttgattgccgtttttaagatccaaccagcatttttagtgatgaacatgcttagcacgagatggacgaaccaagaaaacgtggttctggtcctaggggctgcctttttccaattggcctcagtagatctgcaaataggagttcacggaatcctgaatgccgccgctatagcatggatgattgtccgggcgatcaccttccccacaacctcctccgtcaccatgccagtcttagcgcttctaaccccgggaatgagggctctatacctagatacttacagaatcatcctcctcgttatagggatttgctctctgctgcaagagaggaaaaagaccatggcaaaaaagaaaggagctgtactcttgggcttagcgctcacatccactggatggttttcgcccaccactatagctgccggactaatggtctgcaacccaaacaagaagagagggtggccagctactgagtttttgtcggcagttggattgatgtttgccatcgtaggtggtttggcagagttggatattgaatccatgtcaatacccttcatgctggcaggtctcatggcagtgccctacgtggtgtcaggaaaagcaacagatatgtggcttgaacgggccgccgacatcagctgggagatggatgctgcaatcacaggaagcagtcggaggctggatgtgaagctggatgaagacggagattttcacttgattgatgatcccggtgttccatggaaggtctgggtcctgcgcatgtcttgcattggcttagccgccctcacgccttgggccattgttcccgccgcttttggttattggctcactttaaaaacaacaaaaaaaggaggcgtgttttgggacacgccatccccaaaaccttgctcaaaaggagacaccactacaggagtttaccgcattatggctagagggattcttggcacttaccaggccggcgtcggagtcatgtacgagaatgttttccacacactatggcacacaactagaggagcggccattatgagtggagaaggaaaattgacgccatactggggtagtgtgaaagaagaccgcatagcttacggaggcccatggaggtttgatcgaaaatggaatggaacagatgacgtgcaagtgatcgtggtagaaccggggaaggctgcagtaaacatccagacaaaaccaggggtgtttcggactcccttcggggaggttggggctgttagtctggattacccgcgaggaacatccggctcacccattctggattccaatggagacatcataggcctgtacggcaatggagttgagcttggcgatggttcatacgtcagcgccatcgtgcagggtgaccgtcaggaggaaccagtcccagaagcttacaccccaaacatgttgagaaagagacagatgactgtactagatttgcaccctggttcagggaaaaccaggaaaattctgccacaaataattaaggacgctacccagcagcgcctaagaacagctgtgttggcaccgacgcgggtggtagcagtagaaatggcagaagctttgagagggctcccagtacgatatcaaacttcagcagtgcagagagagcaccaagggaatgaaatagtggatgtgatgtgccacgccactctgacccatagactgatgtcaccgaacagagtgcccaactacaacctatttgtcatggatgaagctcatttcaccgacccagccagtatagctgcacgaggatacattgctaccaaggtggaattaggggaggcagcagccatctttatgacagcgaccccgcctggaaccacggatccttttcctgactcaaatgccccaatccatgatttgcaagatgagataccagacagggcgtggagcagtggatacgaatggatcacagaatatgcgggaaaaaccgtgtggtttgtggcaagcgtaaaaatggggaatgagattgcaatgtgcctccaaagagcggggaaaaaggtcatccaactcaaccgcaagtcctatgacacagaatacccaaaatgtaagaatggagactgggattttgtcatcaccaccgacatctctgaaatgggggccaacttcggtgcgagcagggtcatcgactgtagaaagagcgtgaagcctaccatcttagaagagggagaaggcagagtcatcctcggaaacccatcccccataaccagtgcaagcgcagctcaacggaggggcagggtaggcagaaaccccaaccaggttggagatgaataccactatgggggggccaccagtgaagatgacagtaatctagcccattggacagaggcaaagatcatgttagacaacatacacatgcccaatggactggtggcccagctctatggaccagagagggaaaaggccttcacaatggatggcgaataccgtctcagaggtgaagaaaagaaaaacttcttagagctgcttaggacggctgacctcccggtgtggctggcctacaaggtggcgtccaatggcattcagtacaccgacagaaagtggtgttttgatgggccgcgcacgaatgccatactggaggacaacaccgaggtagagatagtcacccggatgggtgagaggaaaatcctcaagccgagatggcttgatgcaagagtttatgcagatcaccaagccctcaagtggttcaaagacttcgcagcaggaaagagatcagccgttagcttcatagaggtgctcggtcgtatgcctgagcatttcatgggaaagacgcgggaagctttagacaccatgtacttggttgcaacggctgagaaaggtgggaaagcacaccgaatggctctcgaagagctgccagatgcactggaaaccattacacttattgttgctatcactgtgatgacaggaggattctttctactcatgatgcagcggaagggtatagggaagatgggtcttggagctctagtgctcacgctagctaccttcttcctgtgggcggcagaggttcctggaaccaaaatagcagggaccctgctgatcgccctgctgcttatggtggttctcatcccagaaccggaaaaacagaggtcacagacagataaccaactggcggtgtttctcatctgtgtcttgaccgtggttggagtggtggcagcaaacgagtacggaatgctagaaaaaaccaaagcagacctcaagagcatgtttggcggaaagacgcaggcatcaggactgactggattgccaagcatggcattggacctgcgtccagccacagcttgggcactgtatggggggagcacagtcgtgctaacccctcttctgaagcacctgatcacgtcggaatacgtcaccacatcgctagcctcaattaactcacaagctggctcattatttgtcttgccacgaggcgtgccttttaccgacctagacttgaccgttggcctcgtcttccttggctgttggggtcaaatcaccctcacaacgtttttgacagccatggttctggcgacacttcactatgggtacatgctccctggatggcaagcagaagcactcagagctgcccagagaaggacagcggctggaataatgaagaatgccgttgttgacggaatggtcgccactgatgtgcctgaactggaaaggaccactcctctgatgcaaaagaaagtcggacaggtgctcctcataggggtaagcgtggcagcgttcctcgtcaaccccaatgtcaccactgtgagagaagcaggggtgttggtgacggcggctacgctcactttgtgggacaatggagccagtgccgtttggaattccaccactgccccgggactctgccatgtaatgcgaggtagctacctggctggaggctccattgcttggactctcatcaagaacgctgacaagccctccttgaaaaggggaaggcctgggggcaggacgctaggggagcagtggaaggaaaaactaaatgccatgagcagagaagagttttttaaataccggagagaggccataatcgaggtggaccgcactgaagcacgcagggctagacgtgaaaataacatagtgggaggacatccggtttcgcgaggctcagcaaaactccgttggctcgtggagaaaggatttgtctcgccaataggaaaagtcattgatctagggtgtgggcgtggaggatggagctactacgcagcaaccctgaagaaggtccaggaagtcagaggatacacgaaaggtggggcgggacatgaagaaccgatgctcatgcagagctacggctggaacctggtctccctgaagagtggagtggacgtgttttacaaaccttcagagcccagtgacactctgttctgcgacataggggaatcctccccaagtccagaagtagaagaacaacgcacattacgcgtcctagagatgacatctgactggttgcaccgaggacctagagagttctgcataaaagttctttgcccctacatgcccaaggtcatagaaaaaatggaagttctgcagcgccgcttcggaggtgggctagtgcgtctccccctgtcccgcaactccaatcacgagatgtattgggttagtggagccgctggcaatgtggtgcacgctgtgaacatgaccagccaggtactactggggcgaatggatcgcacagtgtggagagggccaaagtatgaggaagatgtcaacctagggagcggaacaagagccgtgggaaagggagaagtccatagcaatcaggagaaaatcaagaagagaatccagaagcttaaagaagaattcgccacaacgtggcacaaagaccctgagcatccataccgcacttggacataccacggaagctatgaagtgaaggctactggctcagctagctctctcgtcaacggagtggtgaagctcatgagcaaaccttgggacgccattgccaacgtcaccaccatggccatgactgacaccaccccgtttggacagcaaagagttttcaaggagaaagttgacacgaaggctcctgagccaccagctggagccaaggaagtgctcaacgagaccaccaactggctgtgggcctacttgtcacgggaaaaaagaccccgcttgtgcaccaaggaagaattcataaagaaagtcaatagcaacgcggctcttggagcagtgttcgctgaacagaatcaatggagcacggcgcgtgaggctgtggatgacccgcggttttgggagatggttgatgaagagagggaaaaccatctgcgaggagagtgtcacacatgtatctataacatgatgggaaaaagagagaagaagcctggagagtttggaaaagctaaaggaagcagggccatttggttcatgtggcttggagcacggtatctagagtttgaagctttggggttcctgaatgaagaccattggctgagccgagagaattcaggaggtggagtggaaggctcaggcgtccaaaagctgggatacatcctccgtgacatagcaggaaagcaaggagggaaaatgtacgctgatgataccgccgggtgggacactagaattaccagaactgatttagaaaatgaagctaaggtgctggagcttctagatggtgaacaccgcatgctcgcccgagccataattgaattgacttacaggcacaaagtggtcaaggtcatgagacctgcagcagaaggaaagaccgtgatggacgtgatatcaagagaagatcaaagggggagtggacaggtggtcacttatgctcttaacactttcacgaacatcgctgtccagctcgtcaggctgatggaggctgagggggtcattggaccacaacacttggaacagctacctagaaaaaacaagatagctgtcaggacctggctctttgagaatggagaggagagagtgaccaggatggcgatcagcggagacgactgtgtcgtcaagccgctggacgacagattcgccacggccctccacttcctcaacgcaatgtcaaaggtcagaaaggatatccaggaatggaagccttcgcatggttggcacgactggcagcaagttcccttctgctctaaccattttcaggagattgtgatgaaagatggaaggagtatcgttgtcccgtgcagaggacaggatgagctgataggcagggctcgcatctccccaggagctggatggaatgtgaaggacacagctcgtctggccaaagcatatgcacagatgtggctactcctatacttccatcgtagggacttgcgtctcatggcaaatgcaatttgctcagcagtgccagtggattgggtgcccacgggcaggacatcctggtcgatacactcgaaaggagagtggatgaccacagaagacatgctgcaggtctggaacagagtctggattgaagaaaatgaatggatgatggacaagactccaatcacaagctggacagacgttccgtacgtgggaaagcgtgaggacatctggtgtggtagcctcatcagaacgcgatccagagcaacctgggctgagaacatctacgcggcgataaaccaggttagagctgtcattgggaaagaaaattatgttgactacatgacctcactcaggagatacgaagacgtcttgatccaggaagacagggtcatctagtgtgatttaaggtagaaaagtagactatgtaaataatgtaaatgagaaaatgcatgcatatggagtcaggccagcaaaagctgccaccggatactgggtagacggtgctgcctgcgtctcagtcccaggaggactgggttaacaaatctgacaatagaaagtgagaaagccctcagaaccgtctcggaagcaggtccctgctcactggaagttgaaggaccaacgtcaggccacaaatttgtgccactccgctggggagtgcggcctgcgcagccccaggaggactgggttaccaaagctgttgagcccccacggcccaagcctcgtctaggatgcaatagacgaggtgtaaggactagaggttagaggagaccccgtggaaacaacaacatgcggcccaagccccctcgaagctgtagaggaggtggaaggactagaggttagaggagaccccgcatttgcatcaagcagcatattgacacctgggaatagactgggagatcttctgctctatctcaacatcagctactaggcacagagcgccgaagtatgtagctggtggtgaggaagaacacaggatct

SEQ ID NO: 3 - DNA, 58 bp, Artificial
  primer
ctcactaaagggaacaaaagctgggtaccgggtctagagaacctgttgattcaacagc

SEQ ID NO: 4 - DNA, 56 bp, Artificial
  primer
caaaagctgggtaccgggcccagatcctgtgttcttcctcaccaccagctacatac

SEQ ID NO: 5 - DNA, 60 bp, Artificial
  Primers
acggccagtgaattgtaatacgactcactatagggcgaattgagttgttagtctacgtgg

SEQ ID NO: 6 - DNA, 60 bp, Artificial
  Primers
tgcgtttgcgttgctcggggtcgtacaccttggaagccattctcttcagtatccctgctg

SEQ ID NO: 7 - DNA, 60 bp, Artificial
  Primers
ggccagtgaattgtaatacgactcactatagggcgaattgtggggaacaattaaaaaatc

SEQ ID NO: 8 - DNA, 60 bp, Artificial
  Primers
tgcgtttgcgttgctcggggtcgtacaccttggaagccataggacacttgtacgtgattg

SEQ ID NO: 9 - DNA, 60 bp, Artificial
  Primers
ggccagtgaattgtaatacgactcactatagggcgaattgtttctcaggcagaatgaacc

SEQ ID NO: 10 - DNA, 55 bp, Artificial
  Primers
tgcgttgctcggggtcgtacaccttggaagccataatcagggctctttggaaatg

SEQ ID NO: 11 - DNA, 60 bp, Artificial
  Primers
cggccagtgaattgtaatacgactcactatagggcgaattgttcatcttactgacagctg

SEQ ID NO: 12 - DNA, 56 bp, Artificial
  Primers
tgcgttgctcggggtcgtacaccttggaagccattttgtcctgctcttcatttagg

SEQ ID NO: 13 - DNA, 60 bp, Artificial
  Primers
acggccagtgaattgtaatacgactcactatagggcgaattgaggttcgtctgcaaacac

SEQ ID NO: 14 - DNA, 54 bp, Artificial
  Primers
tgcgttgctcggggtcgtacaccttggaagccattctcggagagcactccatcg

SEQ ID NO: 15 - DNA, 60 bp, Artificial
  Primers
cggccagtgaattgtaatacgactcactatagggcgaattgacgggcctcgacttcaatg

SEQ ID NO: 16 - DNA, 55 bp, Artificial
  Primers
tgcgttgctcggggtcgtacaccttggaagccatcctcagcctgcacttgagatg

SEQ ID NO: 17 - DNA, 60 bp, Artificial
  Primers
acggccagtgaattgtaatacgactcactatagggcgaattgatggacaaactacagctc

SEQ ID NO: 18 - DNA, 52 bp, Artificial
  Primers
tgcgttgctcggggtcgtacaccttggaagccatcttcaattgtcccggctc

SEQ ID NO: 19 - DNA, 60 bp, Artificial
  Primers
ggccagtgaattgtaatacgactcactatagggcgaattgctcaactggtttaagaaagg

SEQ ID NO: 20 - DNA, 53 bp, Artificial
  Primers
tgcgttgctcggggtcgtacaccttggaagccatatacagcgtcacgactccc

SEQ ID NO: 21 - DNA, 60 bp, Artificial
  Primers
acggccagtgaattgtaatacgactcactatagggcgaattgttgggagttatggtgcag

SEQ ID NO: 22 - DNA, 54 bp, Artificial
  Primers
tgcgttgctcggggtcgtacaccttggaagccatgatgtctcctgtcataatag

SEQ ID NO: 23 - DNA, 60 bp, Artificial
  Primers
ggccagtgaattgtaatacgactcactatagggcgaattgaaaggaatcatgcaggcagg

SEQ ID NO: 24 - DNA, 56 bp, Artificial
  Primers
tgcgttgctcggggtcgtacaccttggaagccatttggccagtggcagcttttaac

SEQ ID NO: 25 - DNA, 18 bp, Artificial
  Primers
atggcttccaaggtgtac

SEQ ID NO: 26 - DNA, 59 bp, Artificial
  Primers
ggccagtgaattgtaatacgactcactatagggcgaattgatggcttccaaggtgtacg

SEQ ID NO: 27 - DNA, 60 bp, Artificial
  Primers
ttaaccctcactaaagggaacaaaagctgggtaccgggcccatcgattttaccacatttg

SEQ ID NO: 28 - DNA, 26 bp, Artificial
  primer
cgaggtgccgtaaagcactaaatcgg

SEQ ID NO: 29 - DNA, 36 bp, Artificial
  primer
cagggccatgaacagcttcagtggtcctcgtccctg

SEQ ID NO: 30 - DNA, 36 bp, Artificial
  Primer
atttaggtgacactatagagttgttagtctacgtgg

SEQ ID NO: 31 - DNA, 60 bp, Artificial
  Primer
agcccgggggatccactagttctagagcggccgccaccgcgggctctgttgtcttttatg

SEQ ID NO: 32 - DNA, 60 bp, Artificial
  Primer
acggccagtgaattgtaatacgactcactatagggcgaattggagctcatttaggtgaca

SEQ ID NO: 33 - DNA, 33 bp, Artificial
  Primer
cagctgacgaagcggttctcacttggaatgctg

SEQ ID NO: 34 - DNA, 34 bp, Artificial
  Primer
tgagaaccgcttcgtcagctgttgtacagtcgac

SEQ ID NO: 35 - DNA, 45 bp, Artificial
  primer
cacttggaatgctgcagggacgaggaccactgaagctgttcatgg

SEQ ID NO: 36 - DNA, 37 bp, Artificial
  primer
ggaatcagcataatgatcaggcctgcagttctgcgtc

SEQ ID NO: 37 - DNA, 37 bp, Artificial
  primer
gacgcagaactgcaggcctgatcattatgctgattcc

SEQ ID NO: 38 - DNA, 37 bp, Artificial
  primer
ggtggtacacgtcccataagtcacccatgtggacgta

SEQ ID NO: 39 - DNA, 37 bp, Artificial
  primer
tacgtccacatgggtgacttatgggacgtgtaccacc

SEQ ID NO: 40 - DNA, 37 bp, Artificial
  primer
ccttagggtggcagattgcttggcttctgtttttatc

SEQ ID NO: 41 - DNA, 37 bp, Artificial
  primer
gataaaaacagaagccaagcaatctgccaccctaagg

SEQ ID NO: 42 - DNA, 39 bp, Artificial
  primer
cctcattgttgtctcaagcattcggccgatagaacttcc

SEQ ID NO: 43 - DNA, 39 bp, Artificial
  primer
ggaagttctatcggccgaatgcttgagacaacaatgagg

SEQ ID NO: 44 - DNA, 37 bp, Artificial
  primer
ccacatcagatttcccagtcttgttactgagcggatt

SEQ ID NO: 45 - DNA, 37 bp, Artificial
  primer
aatccgctcagtaacaagactgggaaatctgatgtgg

SEQ ID NO: 46 - DNA, 24 bp, Artificial
  primer
gtccatctcaagcttacctagatg

SEQ ID NO: 47 - DNA, 24 bp, Artificial
  primer
cccaacacaaacagagcttggaat

SEQ ID NO: 48 - DNA, 24 bp, Artificial
  primer
taagaacagtggggaaacggacac

SEQ ID NO: 49 - DNA, 41 bp, Artificial
  primer
agagaaagaagagaatttaattaactccttggtcacagccg

SEQ ID NO: 50 - DNA, 20 bp, Artificial
  primer
aaaagtcctgagatcaccag

SEQ ID NO: 51 - DNA, 20 bp, Artificial
  primer
atgtcgataaaaaacgaaga

SEQ ID NO: 52 - DNA, 24 bp, Artificial
  primer
tagtataactggtttcatgcagcg

SEQ ID NO: 53 - DNA, 30 bp, Artificial
  primer
tacatctcaactcgagtagagatgggtgag

SEQ ID NO: 54 - DNA, 18 bp, Artificial
  primer
aaggactctgagtgttcg

SEQ ID NO: 55 - DNA, 58 bp, Artificial
  primer
cagtgaattgtaatacgactcactatagggcgaattgggttcgtcgagagaaatatgg

SEQ ID NO: 56 - DNA, 24 bp, Artificial
  primer
ctctaagaagcgtgctccaagcca

SEQ ID NO: 57 - DNA, 24 bp, Artificial
  primer
gggaaaagaatagaaaaaataaaa

SEQ ID NO: 58 - DNA, 24 bp, Artificial
  primer
caggctgcacagtttactcaagct

SEQ ID NO: 59 - DNA, 24 bp, Artificial
  primer
caccatttccatgagttaatcatg

SEQ ID NO: 60 - DNA, 46 bp, Artificial
  Primer
ggaaatctgatgtggaaacagatcacaccagagctgaaccacattc

SEQ ID NO: 61 - DNA, 46 bp, Artificial
  Primer
gaatgtggttcagctctggtgtgatctgtttccacatcagatttcc

SEQ ID NO: 62 - DNA, 37 bp, Artificial
  Primer
aacaccagagctgaaccacattctatcagaaaatgag

SEQ ID NO: 63 - DNA, 38 bp, Artificial
  Primer
atagaatgtggttcagctctggtgttatttgtttccac

SEQ ID NO: 64 - DNA, 74 bp, Artificial
  Primer
ctatagggcgaattggagctcatttaggtgacactatactcgaggaatggtttcatgacctcgctctcccctgg

SEQ ID NO: 65 - DNA, 21 bp, Artificial
  Primer
gaggatgattctgtaagtatc

SEQ ID NO: 66 - DNA, 35 bp, Artificial
  Primer
gatgaacatgcttagcacgagatggacgaaccaag

SEQ ID NO: 67 - DNA, 37 bp, Artificial
  Primer
gcctctgtccaatgggctagattactgtcatcttcac

SEQ ID NO: 68 - DNA, 37 bp, Artificial
  Primer
cggtgcgagcagggtcatcgactgtagaaagagcgtg

SEQ ID NO: 69 - DNA, 20 bp, Artificial
  Primer
gaccaggttccagccgtagc

SEQ ID NO: 70 - DNA, 36 bp, Artificial
  Primer
catagtgggaggacatccggtttcgcgaggctcagc

SEQ ID NO: 71 - DNA, 39 bp, Artificial
  Primer
gcctccatcagcctgacgagctggacagcgatgttcgtg

SEQ ID NO: 72 - DNA, 40 bp, Artificial
  Primer
gctaaggtgctggagcttctagatggtgaacaccgcatgc

SEQ ID NO: 73 - DNA, 56 bp, Artificial
  Primer
ctcactaaagggaacaaaagctgggtaccgggccctgtacagatcctgtgttcttc

SEQ ID NO: 74 - DNA, 37 bp, Artificial
  Primer
ggtggcttcttcgtcatggttatcttccgttctaaaa

SEQ ID NO: 75 - DNA, 36 bp, Artificial
  Primer
ataaccatgacgaagaagccaggagggcccggtaaa

SEQ ID NO: 76 - DNA, 65 bp, Artificial
  Primer
attggagctcatttaggtgacactatagagaagtttatctgtgtgaacttcttggcttagtatcg

SEQ ID NO: 77 - DNA, 49 bp, Artificial
  Primer
ctggctggattgttctcccgatcgctttactggtgcaggagaattttgc

SEQ ID NO: 78 - DNA, 37 bp, Artificial
  Primer
ggttagcttccgttctaaaaaactgtttaaactgcac

SEQ ID NO: 79 - DNA, 59 bp, Artificial
  Primer
gtgcagtttaaacagttttttagaacggaagctaaccatgacgaagaagccaggagggc

SEQ ID NO: 80 - DNA, 36 bp, Artificial
  Primer
cccagagtaggagagttcgatgacaactgttccgtg

SEQ ID NO: 81 - DNA, 49 bp, Artificial
  Primer
ctcctgcaccagtaaagcgatcgggagaacaatccagccagaaaacatc

SEQ ID NO: 82 - DNA, 6 bp, Artificial sequence
  a consensus sequence for the -10 element of a prokaryotic promoter sequence
tataat

SEQ ID NO: 83 - DNA, 6 bp, artificial sequence
  a consensus sequence for the -35 element of a prokaryotic promoter sequence
ttgaca

SEQ ID NO: 84 - DNA, 9 bp, artificial sequence
  a consensus sequence for the extended -10 element of a prokaryotic promoter sequence
tgntataat

SEQ ID NO: 85 - DNA, 21 bp, Dengue virus type 2
ctgacaaagagattctcactt

SEQ ID NO: 86 - DNA, 21 bp, artificial sequence
  an artificial sequence containing one or more silent mutations
ctgacgaagcggttctcactt

SEQ ID NO: 87 - DNA, 21 bp, Dengue virus type 2
ggaccattaaaactgttcatg

SEQ ID NO: 88 - DNA, 21 bp, artificial sequence
  an artificial sequence containing one or more silent mutations
ggaccactgaagctgttcatg

SEQ ID NO: 89 - DNA, 21 bp, Dengue virus type 2
ggaccattaaaactgttcatg

SEQ ID NO: 90 - DNA, 21 bp, artificial sequence
  an artificial sequence containing one or more silent mutations
actgcaggcctgatcattatg

SEQ ID NO: 91 - DNA, 21 bp, Dengue virus type 2
tccacatgggtaacttatggg

SEQ ID NO: 92 - DNA, 21 bp, artificial sequence
  an artificial sequence containing one or more silent mutations
tccacatgggtgacttatggg

SEQ ID NO: 93 - DNA, 33 bp, Dengue virus type 2
atagaaacagaagccaaacaacctgccactcta

SEQ ID NO: 94 - DNA, 33 bp, artificial sequence
  an artificial sequence containing one or more silent mutations
ataaaaacagaagccaaacaatctgccacccta

SEQ ID NO: 95 - DNA, 21 bp, Dengue virus type 2
tctatcggcaaaatgcttgag

SEQ ID NO: 96 - DNA, 21 bp, artificial sequence
  an artificial sequence containing one or more silent mutations
tctatcggcagaatgcttgag

SEQ ID NO: 97 - DNA, 21 bp, Dengue virus type 2
acaagactggaaaatctgatg

SEQ ID NO: 98 - DNA, 21 bp, artificial sequence
  an artificial sequence containing one or more silent mutations
acaagactgggaaatctgatg

SEQ ID NO: 99 - DNA, 21 bp, Dengue virus type 2
acaccagaattgaatcacatt

SEQ ID NO: 100 - DNA, 21 bp, artificial sequence
  an artificial sequence containing one or more silent mutations
acaccagagctgaaccacatt

SEQ ID NO: 101 - DNA, 18 bp, Japanese encephalitis virus
aacggaagataaccatga

SEQ ID NO: 102 - DNA, 18 bp, artificial sequence
  an artificial sequence containing one or more silent mutations
aacggaagctaaccatga

SEQ ID NO: 103 - DNA, 18 bp, Japanese encephalitis virus
atgactaaaaaaccagga

SEQ ID NO: 104 - DNA, 18 bp, artificial sequence
  an artificial sequence containing one or more silent mutations
atgacgaagaagccagga

SEQ ID NO: 105 - DNA, 18 bp, Japanese encephalitis virus
attgggagaacaatccag

SEQ ID NO: 106 - DNA, 18 bp, artificial sequence
  an artificial sequence containing one or more silent mutations
atcgggagaacaatccag